(19) United States
(12) Patent Application Publication
(10) Pub. No.: US 2025/0228710 A1
(43) Pub. Date: Jul. 17, 2025

(54) EARPLUG INSERTION DEVICE LOADING

(71) Applicant: J3 Group, LLC, Lake Oswego, OR (US)

(72) Inventors: Shawn Matthew Johnson, Ridgefield, WA (US); John Matthew Johnson, Ridgefield, WA (US); Gary Nels Anderson, Ridgefield, WA (US); Mark Nels Anderson, Ridgefield, WA (US); Anthony O'Dierno, Lake Oswego, OR (US)

(73) Assignee: J3 Group, LLC, Lake Oswego, OR (US)

(21) Appl. No.: 18/414,044

(22) Filed: Jan. 16, 2024

(51) Int. Cl. A61F 11/12; A61F 11/08; A61F 11/10

(52) U.S. Cl. CPC A61F 11/12; A61F 11/085; A61F 11/10

(57) ABSTRACT

A technique can include pulling an earplug at least partially into a chamber of an earplug loading apparatus. The earplug loading apparatus can include the chamber and a loader. The pulling can include using the loader to pull the earplug relative to the chamber. The earplug loading apparatus can include an earplug insertion device that includes a chamber. The earplug loading apparatus can further include a loader that is structured to load an earplug at least partially into the chamber. The loader can include a grip that is structured to pull an earplug at least partially into the chamber. The loader can be structured to be positioned through the chamber of the earplug insertion device with at least a portion of the loader extending from opposite ends of the chamber at the same time.

### FIELD

The disclosure relates to earplugs such as earplugs in earplug insertion devices.

### BACKGROUND

Foam earplugs have been used to reduce noise in a user's ears by placing an earplug in each of the user's ear canals. Typically, the earplug is inserted into the ear canal by first compressing the earplug by rolling the earplug between the fingers. When the earplug is to be inserted into the ear canal, for example into the left ear, the earplug is held by the left hand while the external ear is drawn upwards and outwards with the right hand, and vice versa if the right ear is concerned. After being inserted, the earplug is kept in place with a fingertip for a few seconds during the expansion thereof inside the ear canal. It can be desirable for the earplug to fill the outer portion of the ear canal and to engage the wall of the ear canal after the expansion. Earplug insertion devices have also been used to insert earplugs into users' ear canals.

### SUMMARY

In one aspect, a technique can include acquiring an earplug loading apparatus. The earplug loading apparatus can include a chamber of an earplug insertion device and a loader. The technique can further include pulling the earplug at least partially into the chamber. The pulling can include using the loader to pull the earplug relative to the chamber.

In another aspect, an earplug loading apparatus can include a loader that can include a grip that is structured to pull an earplug at least partially into a chamber of an earplug insertion device.

In yet another aspect, an earplug loading apparatus can include an earplug insertion device that includes a chamber. The earplug loading apparatus can further include a loader that is structured to load an earplug at least partially into the chamber of the earplug insertion device. The loader can be structured to be positioned through the chamber of the earplug insertion device with at least a portion of the loader extending from opposite ends of the chamber at the same time.

In yet another aspect, an earplug with two opposite ends that are both rounded can be provided and used. Also, an earplug with one rounded end and one flat end can be loaded into an earplug insertion device so that the earplug is inserted into an ear canal with the flat side being inserted first.

This Summary is provided to introduce a selection of concepts in a simplified form. The concepts are further described below in the Detailed Description. This Summary is not intended to identify key features or essential features of the claimed subject matter, nor is it intended to be used to limit the scope of the claimed subject matter. Similarly, the invention is not limited to implementations that address the particular techniques, tools, environments, disadvantages, or advantages discussed in the Background, the Detailed Description, or the attached drawings.

### BRIEF DESCRIPTION OF THE DRAWINGS

FIG. 1 is a right-side view of an earplug loading apparatus that is ready to be positioned to insert an earplug into a user's ear canal. As used herein, this view of the earplug loading apparatus will be referred to as a right-side view or just side view of the earplug loading apparatus (which is determined relative to the loading apparatus chamber) even though in this view it is also a view from the front of the user's ear. Of course, the loading apparatus can be positioned at any of various different orientations relative to a user.

FIG. 2 is a perspective proximal left top perspective view of the loading apparatus of FIG. 1 in a configuration that is ready to load an earplug into the chamber.

FIG. 3 is a grip-end perspective view of an earplug loader from the earplug loading apparatus of FIG. 1.

FIG. 4 is a base-end perspective view of the earplug loader of FIG. 3.

FIG. 5 is a side view of the earplug loader of FIG. 3. The opposite side view is a mirror image of the side view of FIG. 5.

FIG. 6 is a top view of the earplug loader of FIG. 3.

FIG. 7 is a bottom view of the earplug loader of FIG. 3.

FIG. 8 is a grip-end view of the earplug loader of FIG. 3.

FIG. 9 is a base-end view of the earplug loader of FIG. 3.

FIG. 10 is a proximal top left perspective view of the chamber of the earplug loading apparatus of FIG. 1.

FIG. 11 is a distal bottom left perspective view of the chamber of the earplug loading apparatus of FIG. 1.

FIG. 12 is a left-side view of the chamber of FIG. 10. The right-side view is a mirror image of the left-side view.

FIG. 13 is a distal view of the chamber of FIG. 10.

FIG. 14 is a proximal view of the chamber of FIG. 10.

FIG. 15 is a view of the earplug loading apparatus like FIG. 2, but with the earplug held with a grip of the loader.

FIG. 16 is a proximal view of the earplug loading apparatus of FIG. 1, with the loader pulling an earplug into the chamber.

FIG. 17 is a sectional view taken along line 17-17 of FIG. 16.

FIG. 18 is a left-side view of the loading apparatus of FIG. 1 with the loader oriented and inserted in a position to push the earplug from the chamber and into an ear canal.

FIG. 19 is a proximal view of the loading apparatus of FIG. 1 in the configuration of FIG. 18.

FIG. 20 is a sectional view taken along line 20-20 of FIG. 19.

FIG. 21 is a sectional view like FIG. 20 but showing the apparatus in a configuration wherein the loader has been inserted to push the earplug from the chamber so that the earplug is no longer in the chamber.

FIG. 22 is a grip-end perspective view of an alternative loader that can be used with the chamber of FIG. 1 in the same way that the loader of FIG. 1 is used.

FIG. 23 is a base-end perspective view of the loader of FIG. 22.

FIG. 24 is a grip-end perspective view of another alternative loader that can be used with the chamber of FIG. 1 in the same way that the loader of FIG. 1 is used.

FIG. 25 is a base-end perspective view of the loader of FIG. 24.

FIG. 26 is a side view of the loader of FIG. 24. The opposite side view is a mirror image of the side view of FIG. 26.

FIG. 27 is a top view of the loader of FIG. 24.

FIG. 28 is a bottom view of the loader of FIG. 24.

FIG. 29 is a grip-end view of the loader of FIG. 24.

FIG. 30 is a base-end view of the loader of FIG. 24.

FIG. 31 is a grip-end perspective view of another alternative loader that can be used with the chamber of FIG. 1 in the same way that the loader of FIG. 1 is used.

FIG. 32 is a base-end perspective view of the loader of FIG. 31.

FIG. 33 is a side view of the loader of FIG. 31. The opposite side view is a mirror image of the side view of FIG. 33.

FIG. 34 is a top view of the loader of FIG. 31.

FIG. 35 is a bottom view of the loader of FIG. 31.

FIG. 36 is a grip-end view of the loader of FIG. 31.

FIG. 37 is a base-end view of the loader of FIG. 31.

FIG. 38 is a grip-end perspective view of another alternative loader that can be used with the chamber of FIG. 1 in the same way that the loader of FIG. 1 is used.

FIG. 39 is a base-end perspective view of the loader of FIG. 38.

FIG. 40 is a grip-end view of the loader of FIG. 38.

FIG. 41 is a sectional view taken along line 41-41 of FIG. 40.

FIG. 42 is a sectional view like FIG. 41, but with a grip of the loader in a retracted position instead of an extended position illustrated in FIGS. 38-41.

FIG. 43 is a grip-end view of another alternative loader that can be used with the chamber of FIG. 1 in the same way that the loader of FIG. 1 is used. A grip-end perspective view and a base-end perspective view of the loader of FIG. 43 in an extended position would be the same as for the loader of FIGS. 38-39, but the internal features of those loaders are different.

FIG. 44 is a sectional view taken along line 44-44 of FIG. 43.

FIG. 45 is a sectional view taken along line 45-45 of FIG. 43.

FIG. 46 is a sectional view like FIG. 45, but from a perspective viewpoint.

FIG. 47 is a grip-end perspective view of the loader of FIG. 43 with the grip in a retracted position instead of the extended position illustrated in FIGS. 43-46.

FIG. 48 is a grip-end view of the loader of FIG. 43 with the grip in the retracted position of FIG. 47.

FIG. 49 is a sectional view taken along line 49-49 of FIG. 48.

FIG. 50 is a sectional view taken along line 50-50 of FIG. 49.

FIG. 51 is a base end perspective view of the grip of the loader of FIG. 43.

FIG. 52 is a distal perspective view of a plunger that can be used for plunging an earplug from the chamber and into a user's ear canal.

FIG. 53 is a distal view of an earplug insertion device including the plunger of FIG. 51 and the chamber of FIG. 1.

FIG. 54 is a sectional view taken along line 54-54 of FIG. 53.

FIG. 55 is an exploded perspective view of another earplug loading apparatus.

FIG. 56 is a top distal perspective view of a chamber from the earplug loading apparatus of FIG. 55.

FIG. 57 is a bottom distal perspective view of the chamber of FIG. 56.

FIG. 58 is a distal view of the chamber of FIG. 56.

FIG. 59 is a sectional view taken along line 59-59 of FIG. 58.

FIG. 60 is a sectional view taken along line 60-60 of FIG. 58.

FIG. 61 is a top right distal perspective view of a stop from the earplug loading apparatus of FIG. 55.

FIG. 62 is a bottom right proximal perspective view of the stop of FIG. 61.

FIG. 63 is a right-side view of the stop of FIG. 61.

FIG. 64 is a left-side view of the stop of FIG. 61.

FIG. 65 is a top view of the stop of FIG. 59.

FIG. 66 is a distal view of the earplug loading apparatus of FIG. 55 in a configuration without including an earplug.

FIG. 67 is a sectional view taken along line 67-67 of FIG. 66.

FIG. 68 is a sectional view like FIG. 67, but in a different configuration and including an earplug held by the loader of the earplug loading apparatus of FIG. 55.

FIG. 69 is a sectional view like FIG. 67, but in a different configuration after the loader has pulled the earplug into the chamber of the earplug loading apparatus of FIG. 55.

FIG. 70 is a distal view of the earplug loading apparatus of FIG. 55 with an earplug in a different configuration.

FIG. 71 is a sectional view taken along line 71-71 of FIG. 70.

FIG. 72 is a sectional view like FIG. 71, but in a different configuration after the earplug is pushed out of the chamber of the earplug loading apparatus of FIG. 55.

FIG. 73 is a distal-end perspective view of another earplug loading apparatus.

FIG. 74 is a grip-end perspective view of a loader from the earplug loading apparatus of FIG. 73.

FIG. 75 is a base-end perspective view of the loader of FIG. 74.

FIG. 76 is a distal-end perspective view of a chamber from the earplug loading apparatus of FIG. 73.

FIG. 77 is a proximal-end perspective view of the chamber of FIG. 76.

FIG. 78 is a distal-end view of the earplug loading apparatus of FIG. 73.

FIG. 79 is a sectional view taken along line 79-79 of FIG. 78, with the loader of the earplug apparatus positioned through the chamber and grasping an earplug for loading.

FIG. 80 is a sectional view like FIG. 79, but with the earplug loaded in the chamber and with the jaws of the loader being expanded in an expanded section of the chamber.

FIG. 81 is a sectional view like FIG. 79, but with the loader positioned with the jaws within the chamber distally from the expanded section of the chamber, and with an earplug having been inserted into an ear canal (with the ear canal not being illustrated).

FIG. 82 is a distal-end perspective view of another earplug loading apparatus, with the loader of the earplug apparatus positioned through the chamber and grasping an earplug for loading.

FIG. 83 is a proximal-end perspective view of the earplug loading apparatus of FIG. 82.

FIG. 84 is a distal-end view of the earplug loading apparatus of FIG. 82.

FIG. 85 is a sectional view taken along line 85-85 of FIG. 84.

FIG. 86 is a distal-end (and grip-end) perspective view of a loader of the earplug loading apparatus of FIG. 82.

FIG. 87 is a proximal-end perspective view of the loader of FIG. 86.

FIG. 88 is a distal-end perspective view of a chamber of the earplug loading apparatus of FIG. 82.

FIG. 89 is a distal-end view of the chamber of FIG. 86.

FIG. 90 is a sectional view taken along line 88-88 of FIG. 87.

FIG. 91 is a distal-end perspective view of a stop cap of the earplug loading apparatus of FIG. 82.

FIG. 92 is a proximal-end perspective view of the stop cap of FIG. 91.

FIG. 93 is a distal-end perspective view of a stop body of the earplug loading apparatus of FIG. 82.

FIG. 94 is a proximal-end perspective view of the stop body of FIG. 93.

FIG. 95 is a distal-end view of the stop body of FIG. 93.

FIG. 96 is a sectional view taken along line 96-96 of FIG. 95.

FIG. 97 is a sectional view like FIG. 85, but with the earplug loaded in the earplug loading apparatus.

FIG. 98 is a sectional view like FIG. 85, but with the earplug having been inserted into an ear canal (with the ear canal not being illustrated).

FIG. 99 is a distal-end perspective view of another earplug loading apparatus, with the loader of the earplug apparatus positioned through the chamber and grasping an earplug for loading.

FIG. 100 is a proximal-end perspective view of the earplug loading apparatus of FIG. 99.

FIG. 101 is a distal-end view of the earplug loading apparatus of FIG. 99.

FIG. 102 is a sectional view taken along line 102-102 of FIG. 101.

FIG. 103 is a distal-end (and grip-end) perspective view of a loader of the earplug loading apparatus of FIG. 99.

FIG. 104 is a proximal-end perspective view of the loader of FIG. 103.

FIG. 105 is a distal-end perspective view of a chamber of the earplug loading apparatus of FIG. 99.

FIG. 106 is a distal-end view of the chamber of FIG. 105.

FIG. 107 is a sectional view taken along line 107-107 of FIG. 106.

FIG. 108 is a distal-end perspective view of a stop cap of the earplug loading apparatus of FIG. 99.

FIG. 109 is a proximal-end perspective view of the stop cap of FIG. 91.

FIG. 110 is a distal-end perspective view of a stop body of the earplug loading apparatus of FIG. 99 with a ring.

FIG. 111 is a proximal-end perspective view of the stop body of FIG. 110 with the ring.

FIG. 112 is a distal-end view of the stop body and ring of FIG. 110.

FIG. 113 is a sectional view taken along line 113-113 of FIG. 112.

FIG. 114 is a sectional view like FIG. 102, but with the earplug loaded in the earplug loading apparatus.

FIG. 115 is a sectional view like FIG. 114, but with the earplug having been inserted into an ear canal (with the ear canal not being illustrated).

FIG. 116 is a sectional view like FIG. 79, but with the earplug rotated to an opposite orientation with a rounded end facing proximally and a flat end facing distally.

FIG. 117 is a sectional view like FIG. 80, with the earplug rotated to an opposite orientation with a rounded end facing proximally and a flat end facing distally.

FIG. 118 is a section view like FIG. 117 but illustrating an earplug that is rounded on its distal and proximal ends.

FIG. 119 is a flowchart of an earplug loading and insertion technique.

The description and drawings may refer to the same or similar features in different drawings with the same reference numbers.

### DETAILED DESCRIPTION

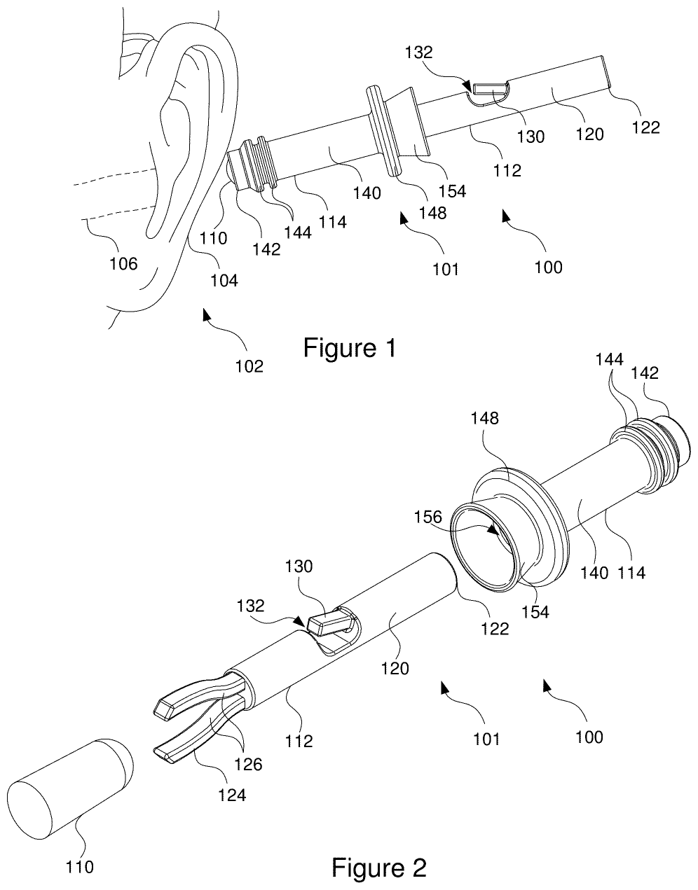

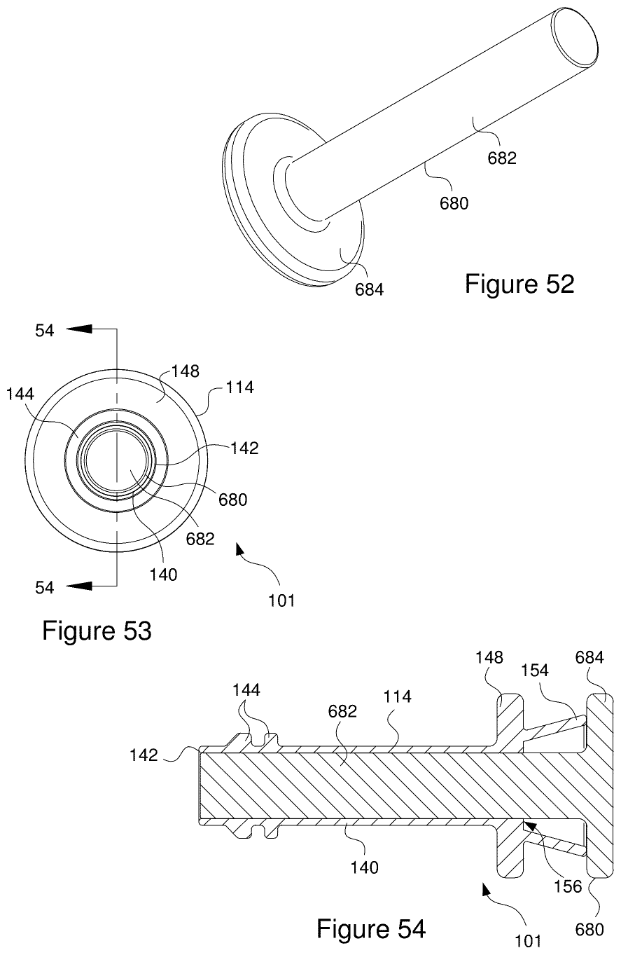

Referring to FIG. 1, an earplug loading apparatus 100 is illustrated. As discussed below, the earplug loading apparatus 100 can include an earplug insertion device 101. The earplug insertion device may include the same components as the earplug loading apparatus (as where the loader discussed below also acts as a plunger for pushing an earplug out of the chamber). However, the earplug insertion device may include a separate plunger (such as the plunger discussed below with reference to FIGS. 52-54) instead of (or in addition to) the loader discussed herein as part of the earplug loading apparatus. Also illustrated is a portion of a user 102 including an ear 104 around an ear canal 106 (shown in dashed lines). The earplug loading apparatus 100 is located near the ear canal 106 and is oriented to be moved to an opening of the ear canal 106 to insert an earplug into the ear canal 106 in a manner that is discussed in more detail below.

### I. Example Earplug Loading Apparatus

Referring now to FIGS. 1-2, the earplug loading apparatus 100 can include an earplug 110, a loader 112, and a chamber 114. The loader 112 can be structured to load the earplug 110 (and/or some other earplug) into the chamber 114 (i.e., at least partially in the chamber 114, and possibly fully in the chamber 114). The loader 112 can also be structured to push the earplug 110 out of the chamber 114 and into the ear canal 106 of the user 102 in an earplug insertion operation.

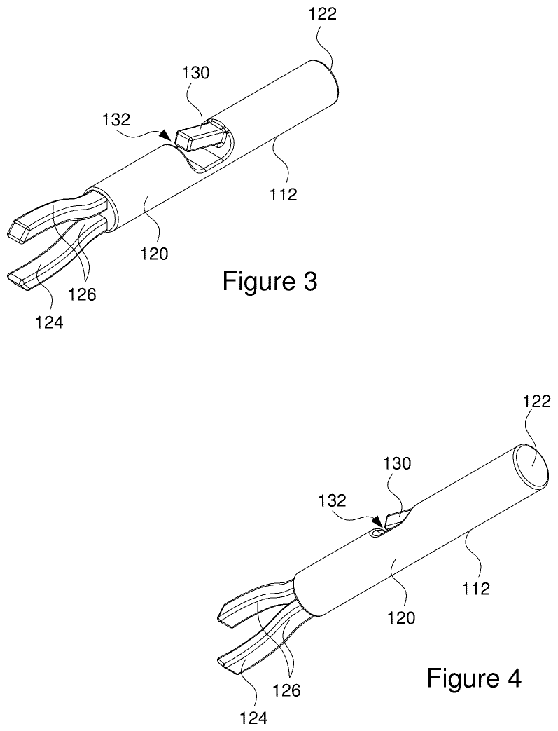

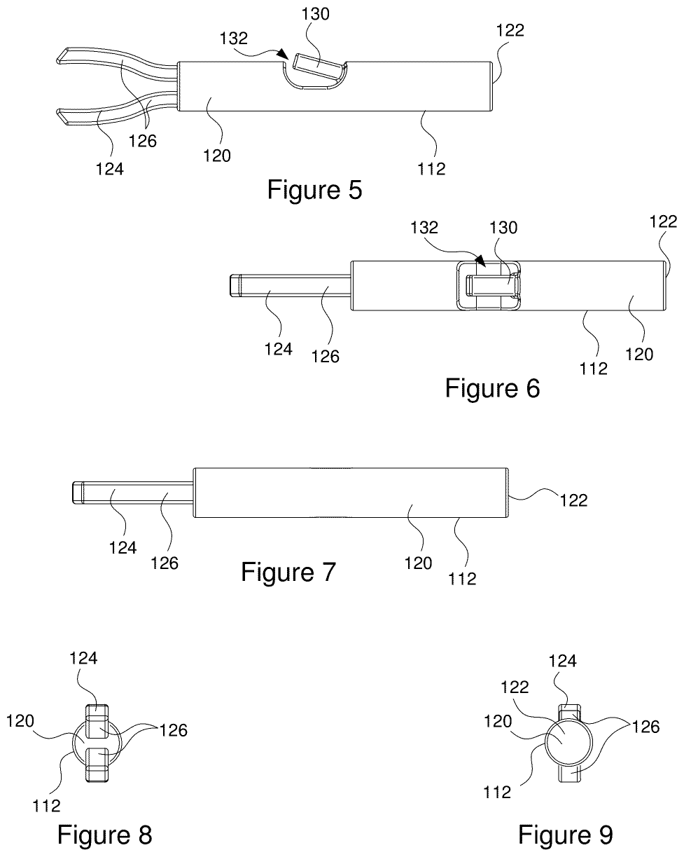

Referring now to FIGS. 3-9, the loader 112 will be discussed in more detail. The loader 112 can include a loader body 120 that can be sized to fit into the chamber 114. For example, the loader body 120 can be a generally cylindrical body. The loader body 120 can extend from a loader base 122 to a grip 124, with a grip direction being axially toward a terminus of the grip 124 and a base direction being axially toward the loader base 122. As an example, the grip 124 can include a pair of opposing jaws 126 that extend from loader body 120 in a curved manner. For example, each jaw 126 may curve outwardly away from the other jaw 126 and then back inwardly toward the other jaw 126 as the jaw extends in the grip direction away from the loader body 120. The loader body may be some other shape, such as where at least a portion of the loader body has a generally plus (+) shaped cross section or some other non-circular cross section instead of the round cross section of a cylinder. Loader 112 can also have a catch 130, which can be a cantilever beam that extends from a wall of an aperture such as a cutout 132 defined in the loader body 120. The catch 130 can angle in the grip direction and radially out so that at least a portion of a terminus of the catch 130 protrudes radially out farther than an outer periphery of the loader body 120.

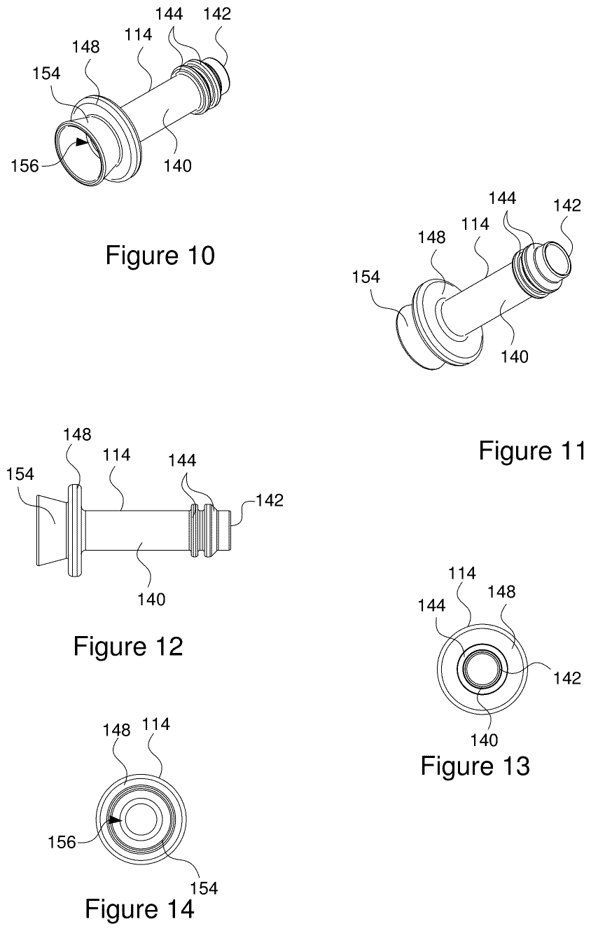

Referring now to FIGS. 10-14, the chamber 114 will be discussed in more detail. As used herein, the terms distal and proximal can refer to directions relative to the chamber 114, with a distal direction being along an axis of the chamber toward a tip of the chamber 114 and a proximal direction being along an axis of the chamber opposite the distal direction. The chamber 114 can include a chamber body 140 that can be a generally hollow cylindrical shape defining a generally cylindrical internal space therein in which the earplug 110 can be loaded, so that the earplug 110 can later be plunged from the chamber 114 into an ear canal 106. The chamber body 140 can include a distal tip 142 that can be sized to fit into a typical ear canal opening. Also, different chambers 114 can be made in different sizes for different sized ear canals and ear canal openings. The chamber 114 can include tip flanges 144, which can be rings extending radially out from the chamber body 140. The tip flanges 144 can be located adjacent to a proximal end of the distal tip 142, still leaving a portion of the distal tip 142 extending distally from the distal-most tip flange 144. The tip flanges 144 can be sized so that they do not fit easily into a typical ear canal opening, to inhibit insertion of the distal tip 142 too far into the ear canal 106. The distal-most tip flange 144 can include a beveled distal surface to increase the comfort of the device as it is positioned in the opening of the ear canal 106.

At a proximal end of the chamber body 140, a base flange 148 can extend radially out from the chamber body 140. For example, the base flange 148 can be a circular radial wall. Additionally, a funnel 154 can angle radially out as it extends in the proximal direction from a proximal side of the base flange 148. The base flange 148 can include a catch surface or catch area 156, which can be a proximally facing area at an inner portion of an annular surface extending radially in from the distal end of the funnel 154.

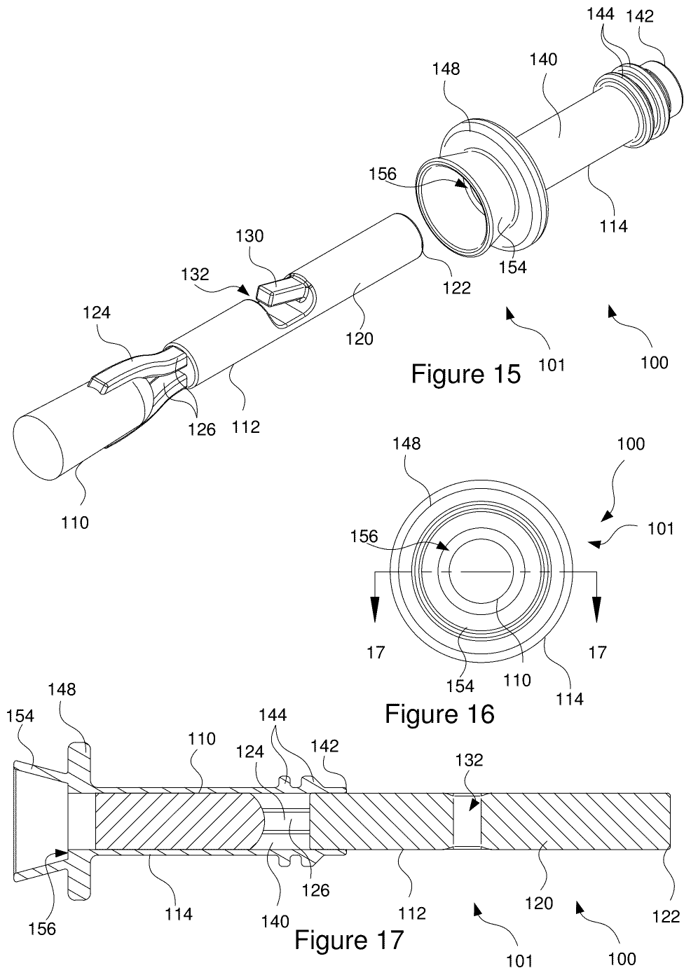

Use of the earplug loading apparatus 100 will now be discussed. Referring to FIG. 15, the earplug 110 can be placed in the grip 124 of the loader 112 between the jaws 126 so that the grip 124 holds the earplug 110. For example, a distal end of the earplug 110 (the end that will be pushed deepest into the ear canal 106) can be positioned between the jaws 126 with the remainder of the earplug 110 extending proximally therefrom. The loader 112 can be axially aligned with the chamber 114 with the grip 124 extending in the proximal direction from the loader body 120. Loader base 122 can then be inserted into the proximal end of the chamber 114, followed by the loader body 120. As the loader body 120 passes into the chamber 114, the inner edge of the base flange 148 of the chamber 114 can press the catch 130 of the loader 112 radially in because of the slope of the catch 130. Thus, as the loader 112 passes into the chamber 114 with the loader base 122 leading into the chamber 114, the catch 130 can proceed into the loader without catching on the chamber 114.

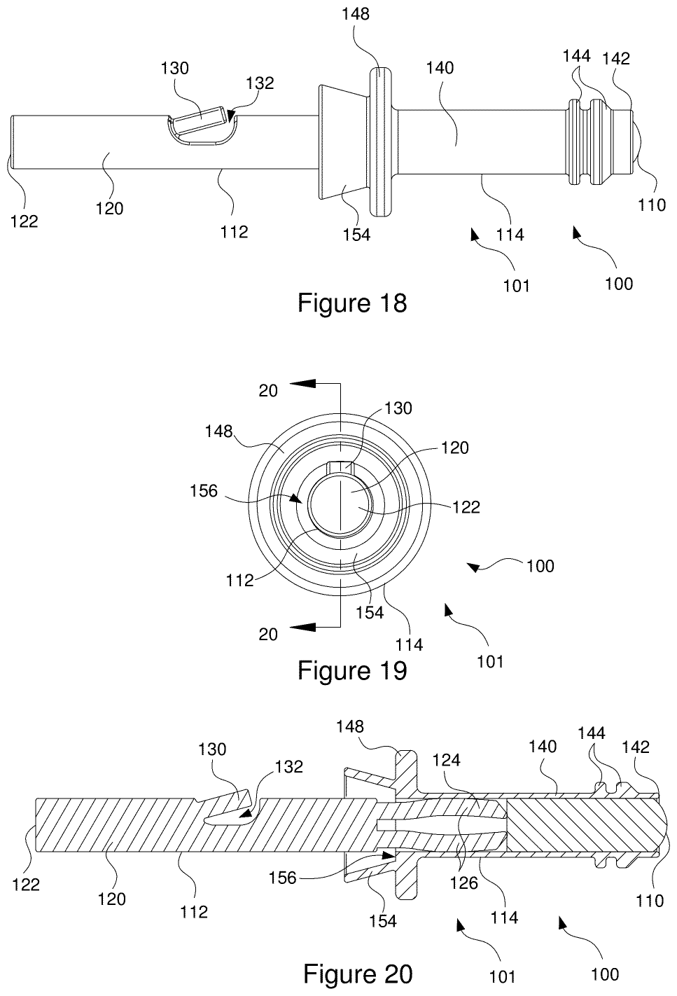

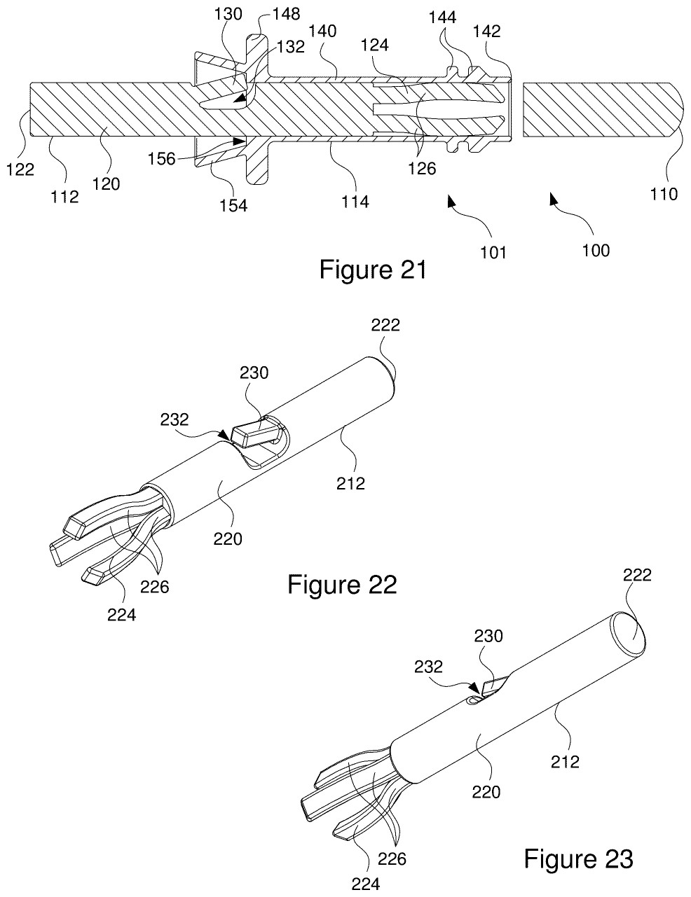

As the loader 112 continues through the chamber 114, the funnel 154 and/or the inner edge of the base flange 148 of the chamber 114 can press the jaws 126 of the grip 124 radially in because of the slope of the jaws 126. As the jaws 126 are thereby squeezed toward each other, the jaws 126 can squeeze and hold or grip the earplug 110 more tightly, allowing the jaws 126 to grip the earplug 110 tightly enough to pull the earplug 110 into the interior space of the chamber body 140. The funnel 154 can help to compress the earplug 110, preparing the earplug 110 to enter the interior space of the chamber body 140. FIGS. 16-17 illustrate the earplug loading apparatus 100 as the loader 112 pulls the earplug in the distal direction through the chamber body 140. As the jaws 126 of the loader 112 begin to exit the distal tip 142 of the chamber body 140, the jaws 126 can extend away from each other as the jaws 126 exit the chamber body 140 due to a spring action from the jaws, which can be biased toward their open position illustrated in FIGS. 2-9. This can result in the jaws 126 loosening their grip on the earplug 110. Once the grip of the jaws 126 on the earplug 110 is loose enough, the jaws 126 can continue moving in the distal direction without pulling the earplug 110, so that the earplug 110 remains in the chamber 114, such as in the position illustrated in FIGS. 18-20.

Besides illustrating a possible loaded position of the earplug 110 in the chamber 114, FIGS. 18-20 illustrate the loader 112 in position to push the earplug 110 out of the chamber 114 and into the ear canal 106. (This is the same configuration of the earplug loading apparatus 100 as is illustrated in FIG. 1.) With the earplug loading apparatus 100 in the configuration of FIGS. 1 and 18-20, the earplug loading apparatus 100 can be moved and oriented so that the chamber 114 and the loader 112 are aligned with the ear canal 106 and the distal tip 142 is positioned in the opening of the ear canal 106. With the earplug loading apparatus 100 in that configuration, the loader 112 can be pushed distally relative to the chamber 114 while the chamber 114 remains in the same position relative to the ear canal 106. With this movement, the tips of the jaws 126 of the loader 112 can push against the earplug 110 to push the earplug out of the distal tip 142 of the chamber body 140 and into the ear canal 106. The earplug 110 can then expand until the earplug 110 is seated in the ear canal 106 for reduction of noise for the user 102. Accordingly, the earplug loading apparatus 100 can be used to load the earplug into the chamber 114 and to insert the earplug into a user's ear canal 106.

The earplug loading apparatus 100 can be reused in this same manner to load another earplug 110 into the chamber 114 and to eject the earplug 110 from the chamber 114 and into an ear canal (such as the other ear canal of the user 102, an ear canal of a different user, or even an earplug into the same ear canal 106 at a different time).

The components of the earplug loading apparatus 100 can be made of materials that are appropriate for the functions and structures described herein. For example, the loader 112 and the chamber 114 may be made of polymer materials, composite materials, and/or metals. For example, the materials may be appropriately strong, stiff/flexible, and durable. The loader 112 and/or the chamber 114 can be formed with manufacturing techniques such as molding, milling, lathing, and/or additive manufacturing such as 3D printing. The earplug 110 can be made of a compliant material such as foam, and they can be formed by manufacturing processes such as molding.

### II. Additional Loader Examples

Several examples of different loaders that can be used with the other components of the earplug loading apparatus 100 will now be discussed.

Referring to FIGS. 22-23, another loader 212 will be discussed. The loader 212 can include a loader body 220, a loader base 222, a grip 224 with three opposing jaws 226, a catch 230, and a defined cutout 232. The loader body 220, the loader base 222, the catch 230, and the defined cutout 232 can be the same as for the loader 112 discussed above. The grip 224 can include the three jaws 226, which can each be the same as one of the jaws 126 in the loader 112 discussed above. However, the three jaws 226 can be circumferentially spaced around an axis of the loader body 220, such as being equally spaced so that the centers of the jaws 226 are one hundred and twenty degrees apart. The loader 212 can be used in the same way as the loader 112 discussed above. The loader 212 can be made of appropriate materials using appropriate manufacturing techniques such as those discussed above.

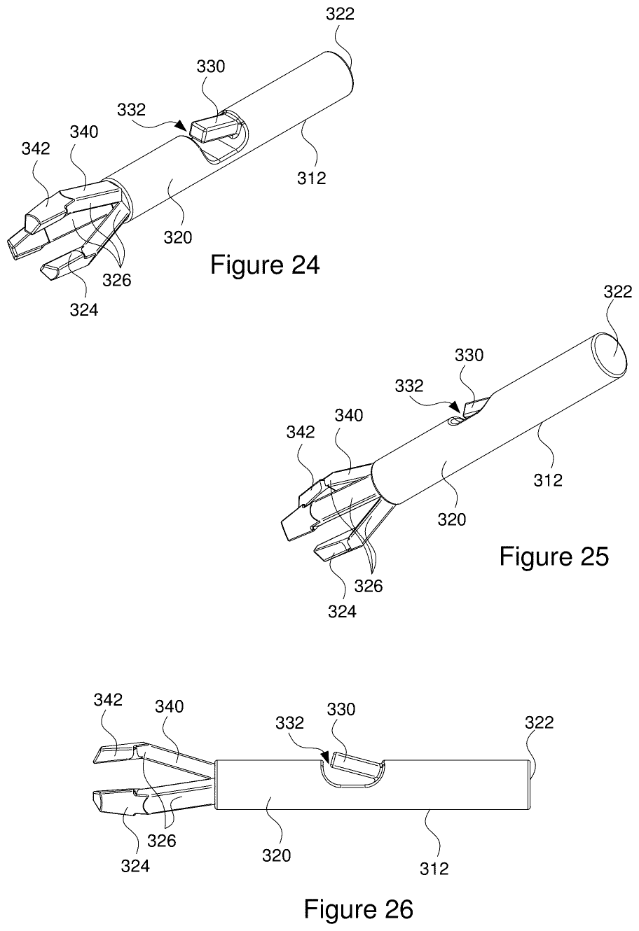

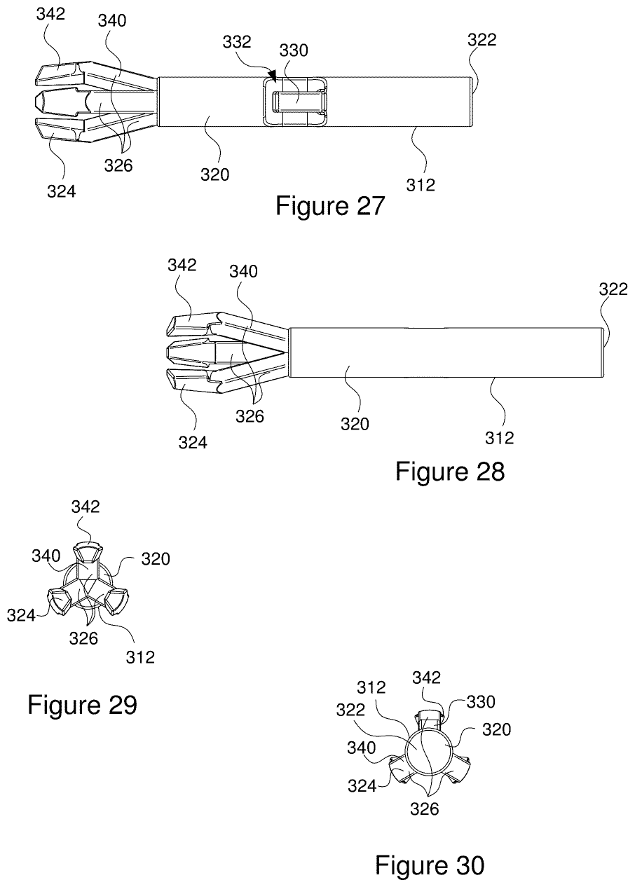

Referring now to FIGS. 24-30, an example of another loader 312 will be discussed. The loader 312 can include a loader body 320, a loader base 322, a grip 324 with three opposing jaws 326, a catch 330, and a defined cutout 332. The loader body 320, the loader base 322, the catch 330, and the defined cutout 332 can be the same as for the loader 112 discussed above. The grip 324 can include the three jaws 326, which can each be different from the jaws 126 in the loader 112 discussed above. Specifically, each jaw 326 can include a first section 340 that angles radially outward as it extends in the grip direction to meet a second section 342 that angles radially inward as it extends further in the grip direction to a tip or terminus of the jaw. The second section 342 of each jaw 326 can include a portion that is wider toward the outer surface of the second section 342, and it can narrow as it extends inward toward an axis of the loader 312. Also, the outer surface of the second section 342 can broaden as it extends in the base direction away from the tip or terminus of the jaw 326, and the jaw 326 may then narrow again as it extends further in the base direction away from the tip or terminus of the jaw 326. The three jaws 326 can be circumferentially spaced around the axis of the loader body 320, such as being equally spaced so that the centers of the jaws 226 are one hundred and twenty degrees apart. The loader 312 can be used in the same way as the loader 112 discussed above. Also, another loader example may have only two jaws that are like the jaws 326 of the loader 312 of FIGS. 24-30. Also, other examples of loaders may include two jaws, three jaws, or more than three jaws that are like any of the loader jaws discussed and illustrated herein. The loader 312 can be made of appropriate materials using appropriate manufacturing techniques such as those discussed above.

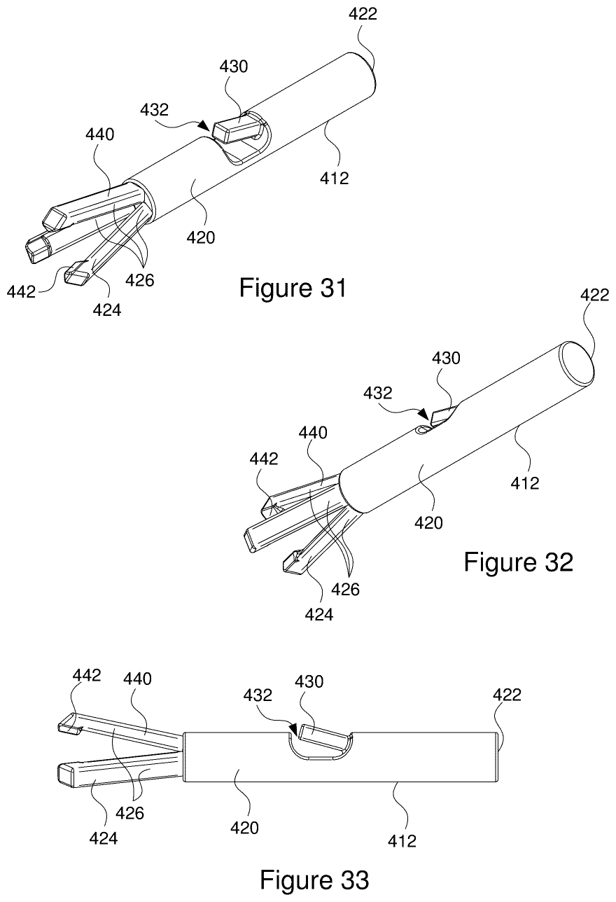

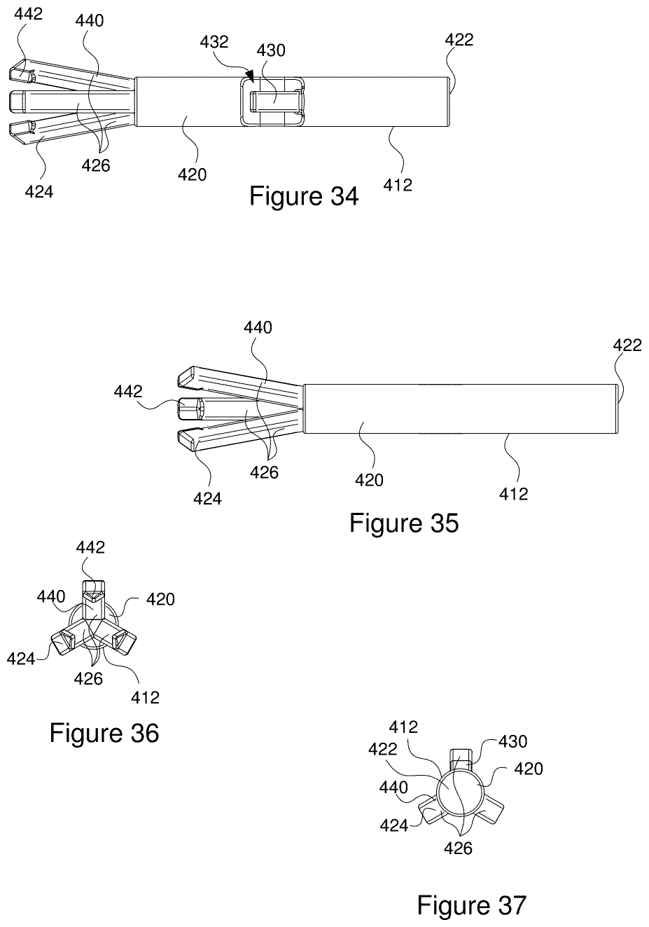

Referring now to FIGS. 31-37, an example of another loader 412 will be discussed. The loader 412 can include a loader body 420, a loader base 422, a grip 424 with three opposing jaws 426, a catch 430, and a defined cutout 432. The loader body 420, the loader base 422, the catch 430, and the defined cutout 432 can be the same as for the loader 112 discussed above. The grip 424 can include the three jaws 426, which can each be different from the jaws 126 in the loader 112 discussed above. Specifically, each jaw 426 can include a first section 440 that angles radially outward as it extends in the grip direction to a tip or terminus of the jaw 426. A second section 442 can be a protrusion or knob that extends radially in from the first section 440 near the tip or terminus of the jaw 426. The second section 442 of each jaw 426 can become narrower as it extends radially inward toward the axis of the loader body 420. The three jaws 426 can be circumferentially spaced around the axis of the loader body 420, such as being equally spaced so that the centers of the jaws 426 are one hundred and twenty degrees apart. The loader 412 can be used in the same way as the loader 112 discussed above. Also, another loader example may have only two jaws or more than three jaws that are like the jaws 426 of the loader of FIGS. 24-30. The loader 412 can be made of appropriate materials using appropriate manufacturing techniques such as those discussed above.

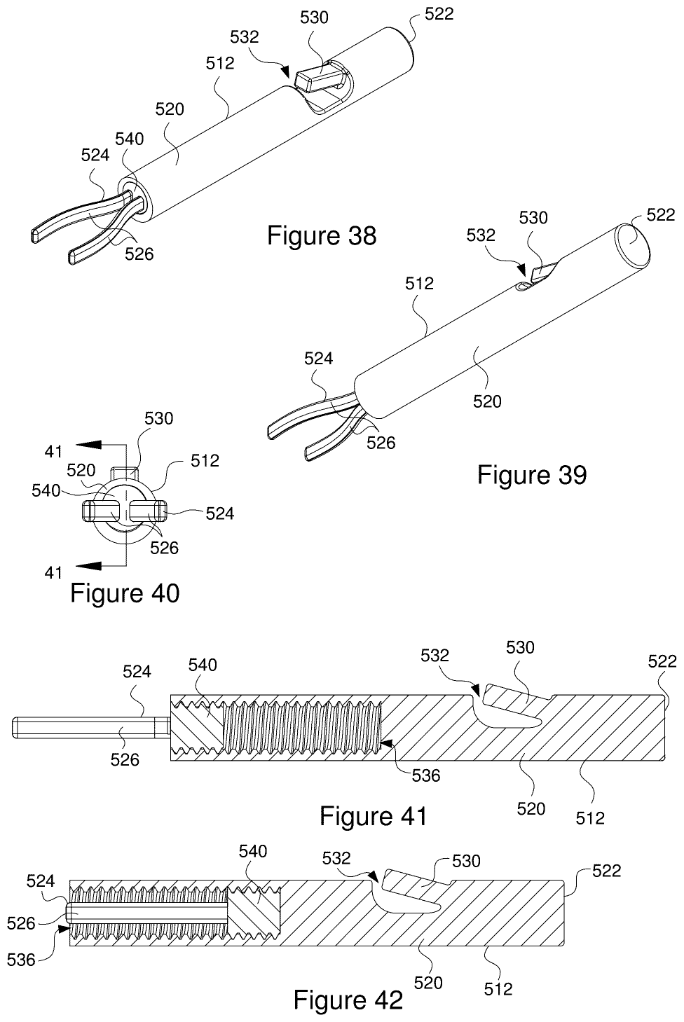

Referring now to FIGS. 38-42, an example of another loader 512 will be discussed. The loader 512 can include a loader body 520, a loader base 522, a grip 524 with two opposing jaws 526, a catch 530, and a defined cutout 532. The loader body 520 can be like the loader body 120 of the loader 112 discussed above, except that the loader body 520 may be longer than the loader body 120, extending farther in the grip direction. Also, the loader body 520 can define an internally threaded cavity 536 extending into the loader body 520 in the base direction from the jaw end of the loader body 520. The jaws 526 can each have cross sections that are thinner than the jaws 126 of the loader 112 discussed above. Also, in addition to the jaws 526, the grip 524 can include a grip base 540 that is externally threaded and to which the jaws 526 can be secured. Specifically, the jaws 526 can extend in the jaw direction from the grip base 540, curving radially outward as they extend from the grip base 540 and then curving back radially inward toward each other as they extend in the grip direction to tips or terminuses of the jaws 526.

The grip base 540 can be threaded into the internally threaded cavity 536 in the loader body 520. The grip 524 can be turned relative to the loader body to screw the grip between an extended position illustrated in FIGS. 38-41 wherein a grip-end surface of the grip base 540 can be flush with the grip-end surface of the loader body 520, and a retracted position illustrated in FIG. 52 wherein the grip base 540 is seated against a base end of the internally threaded cavity 536. The threads of the grip base 540 and the internally threaded cavity 536 may be different from those illustrated in the figures. For example, the threads may be courser and/or have steeper angles than the illustrated threads so that fewer turns of the grip 524 are needed to retract and extend the grip 524.

In use, the loader 512 can be used similarly to the loader 112 discussed above. However, the grip 524 can be turned to the extended position prior to gripping an earplug and pulling the earplug into the chamber 114 to load the earplug in the chamber 114. The grip 524 can be turned to the retracted position prior to using the loader 512 to push an earplug from the chamber 114 and into an ear canal. Thus, for plunging or pushing an earplug from the chamber 114, the tips of the jaws 526 and the grip end of the loader body 520 can all push on the earplug to push the earplug out of the chamber 114 and into an ear canal. Thus, the extended position of the grip 524 may be termed a loading position and the retracted position of the grip 524 may be termed a plunging position or a pushing position.

Components of the loader 512 can be made of appropriate materials using appropriate manufacturing techniques such as those discussed above. To assemble the loader, the grip base 540 can be threaded into the cavity 536 with the jaws 526 extending in the grip direction from the grip base 540.

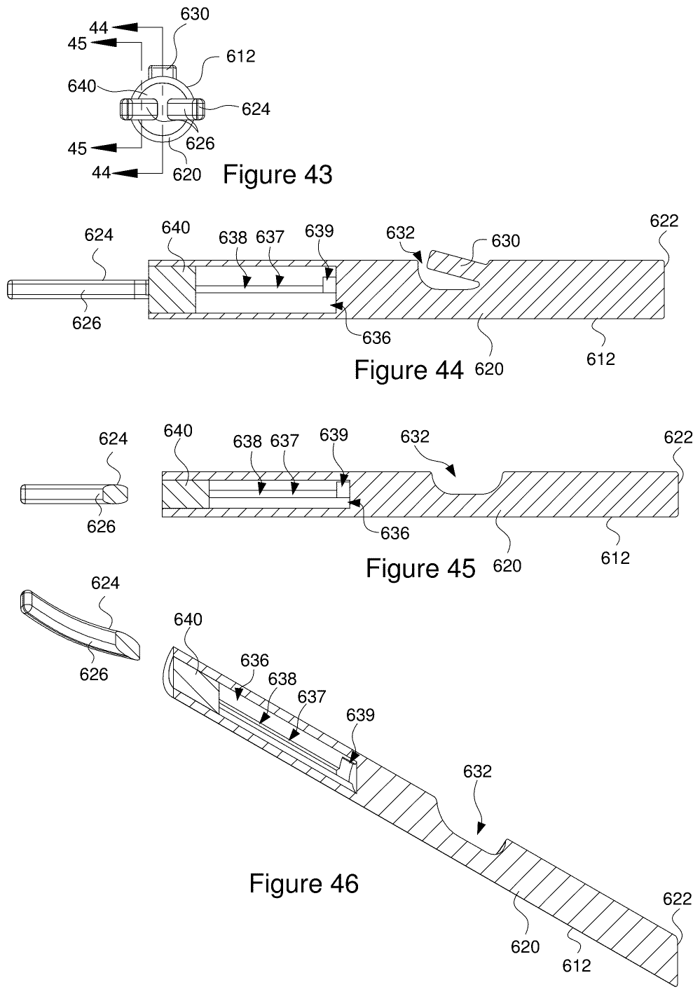

Referring now to FIGS. 43-51, an example of another loader 612 will be discussed. The loader 612 can include a loader body 620, a loader base 622, a grip 624 with two opposing jaws 626, a catch 630, and a defined cutout 632. The loader body 620 can be like the loader body 520 discussed above, except that the loader body 620 can define a slotted cavity 636 instead of the threaded cavity 536. The slotted cavity 636 can be like the threaded cavity 536 except the slotted cavity 636 can include slots 637 instead of the threads of the threaded cavity 536.

The slots 637 can include two slots that are opposite each other (i.e., spaced one-hundred and eighty degrees apart, around the axis of the loader body 520). The slots 637 can each extend radially outward and into the loader body 620 surrounding the slotted cavity 636. Each slot 637 can include an axially extending section 638 that extends from the grip end of the loader body 620, extending in the base direction to a circumferentially extending section 639 adjacent to the base end of the slotted cavity 636. Each circumferentially extending section 639 can extend circumferentially from the axially extending section 638, with the circumferentially extending sections 639 for both slots 637 extending in the same circumferential direction from their respective axially extending sections 638 (such as the circumferentially extending sections 639 of both slots 637 extending in a counterclockwise direction from the respective axially extending section 638, when considered from the perspective of someone looking in the grip direction (from the base end to the grip end of the loader 612, as in the line of sight for FIG. 50)). The slots 637 can extend partially through the walls of the loader body 620 as illustrated, or all or part of the slots 637 may extend fully through the walls of the loader body 620.

The grip 624 can include a grip base 640 and the jaws 626, which can be like the grip 524 except the grip base 640 can include guides 642 instead of external threads. The guides 642 can extend radially out from opposite sides of the grip base 640. The guides 642 can be shaped and positioned to allow the guides to extend into the slots 637 and to slide axially along the axially extending sections of the slots and circumferentially along the circumferentially extending sections of the slots 637. The guides 642 can each include a face closest to the base end of the grip base 640 (the end opposite the jaws 626) that slopes in the base direction and radially inward toward a cylindrical body of the grip base 640 so that these faces of the guides 642 form ramps or wedges. The grip base 640 can define cutouts or apertures of material around the guides 642, which can allow the guides 642 to bend radially inwardly when pressure is applied to the guides and then spring back out when the pressure is released. Examples of such cutouts are illustrated in the perspective view FIG. 51 but are now shown in the sectional view of FIG. 50.

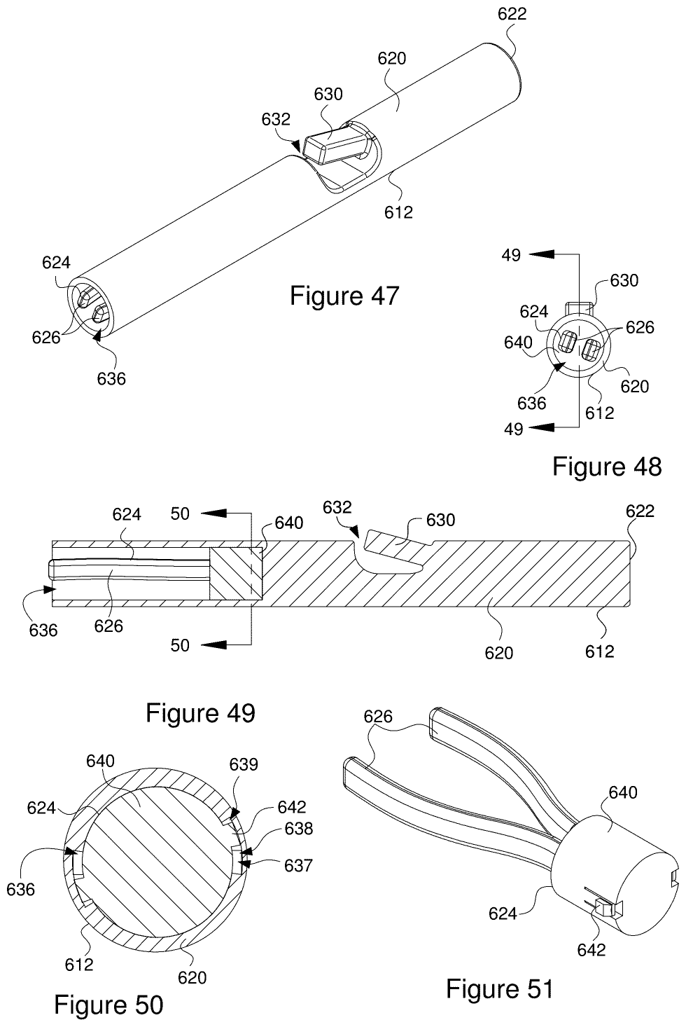

The grip 624 can be moved between an extended position illustrated in FIGS. 43-46 (the extended position for the loader 612 can also look the same externally as the loader 512 illustrated in FIGS. 38-40 above) and a retracted position illustrated in FIGS. 47-50. In the extended position, the grip 624 can be positioned with the guides 642 in the grip ends of the axially extending sections of the slots 637. Because the slots 637 do not extend in the grip direction to all the way to the grip end of the loader body 620, contact between the grip ends of the guides 642 and walls of the loader body 620 at the grip ends of the slots 637 can inhibit movement of the grip base 640 out of the loader body 620, even when the loader 612 is being used to pull an earplug held in the jaws 626 of the grip 624. To move from the extended position to the retracted position, the grip 624 can be pushed axially in the base direction into the cavity 636, with the guides 642 sliding along the axially extending sections 638 of the slots 637 until the guides 642 reach the circumferentially extending sections 639 of the slots 637. The grip 624 can then be rotated in the direction of the circumferentially extending sections 639 of the slots 637 so that the guides 642 become seated in the circumferentially extending sections 639 of the slots 637. In that position, the material of the loader body 620 around the circumferentially extending sections 639 of the slots can inhibit axial movement of the grip 624. The grip 624 can be moved in the opposite directions relative to the loader body 620 (circumferentially and then axially) to move the grip 624 back to the extended position illustrated in FIGS. 43-46.

In use, the loader 612 can be used similarly to the loader 512 discussed above. The grip 624 can be slid to the extended position prior to gripping an earplug and pulling the earplug into the chamber 114 to load the earplug in the chamber 114. The grip 624 can be slid to the retracted position prior to using the loader 612 to push an earplug from the chamber 114 and into an ear canal. Thus, for plunging or pushing an earplug from the chamber 114, the tips of the jaws 626 and the grip end of the loader body 620 can all push on the earplug to push the earplug out of the chamber 114 and into an ear canal. Thus, the extended position of the grip 624 may be termed a loading position and the retracted position of the grip 624 may be termed a plunging position, a pushing position, or an earplug insertion position.

Components of the loader 612 can be made of appropriate materials using appropriate manufacturing techniques such as those discussed above. To assemble the loader, the grip base 640 can be oriented so that the guides 642 are aligned with the slots 637, with the jaws 626 extending in the grip direction from the grip base 640. In this orientation, the grip base 640 can be pushed into the cavity 636. The sloped base-end surfaces of the guides 642 can act as wedges to push the adjacent portions of the loader body 620 outward and/or push the guides 642 radially inward (especially if cutouts are defined around the guides as illustrated in FIG. 51), allowing the guides 642 to pass axially into the slots 637. With the guides 642 positioned in the slots 637, the adjacent portions of the loader body 620 can spring back inward, inhibiting movement of the grip base 640 back out of the loader body 620. Alternatively, the components of the loader 612 can be formed with the grip base 640 already positioned in the loader body 620 using additive manufacturing techniques such as 3D printing.

### III. Plunger Example

Referring now to FIGS. 52-54, an example of a plunger 680 will be discussed. The plunger 680 can be used for plunging or pushing an earplug out of the chamber 114 instead of using a loader to push the earplug out of the chamber 114. The plunger 680 can include a plunger body 682 that can be cylindrical. However, as with the loader bodies discussed here, the plunger body 682 could have a different cross-sectional shape, such as a plus (+) shaped cross section for at least part of the plunger body. The plunger 680 can also include a plunger flange 684 that can extend radially out from one end of the cylindrical plunger body 682. In use, the end of the plunger body 682 opposite the plunger flange 684 can be inserted into the opening at the proximal end of the chamber 114. The plunger 680 can then be pushed in the distal direction relative to the chamber 114 to push an earplug 110 out of the distal tip 142 of the chamber 114. The plunger 680 can then be pulled in the proximal direction relative to the chamber 114 to remove the plunger 680 from the chamber 114, allowing a loader to be used to load another earplug in the chamber 114. Thus, the plunger 680 can be a component in the earplug insertion device 101 with the chamber 114, which can be part of an earplug loading apparatus 100 along with a loader. The plunger 680 can be made of appropriate materials using appropriate manufacturing techniques such as those discussed above.

IV. Additional Earplug Loading Apparatus Example with Stop

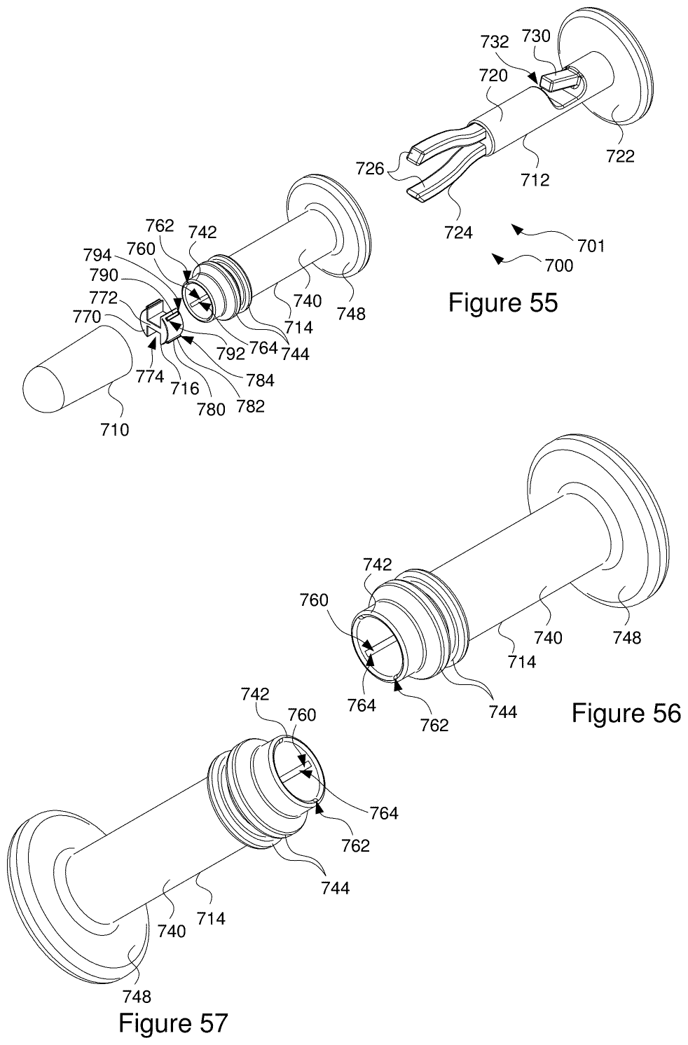

Referring now to FIG. 55, another example of an earplug loading apparatus 700 will be discussed. As discussed below, the earplug loading apparatus 700 can include an earplug insertion device 701 (where the loader of the earplug loading apparatus 700 can act as a plunger for the earplug insertion device 701, or a separate plunger like the plunger discussed above can be part of the earplug insertion device 701 and can be used in place of the earplug loader for plunging or pushing an earplug out of the chamber). The earplug loading apparatus 700 can include an earplug 710, a loader 712, a chamber 714, and a stop 716. The loader 712 can be structured to load the earplug 710 (and/or some other earplug) into the chamber 714 (i.e., at least partially in the chamber 714, and possibly fully in the chamber 714). The loader 712 can also be structured to push the earplug 710 out of the chamber 714 and into the ear canal of the user (like the ear canal 106 of the user 102 discussed above) via the stop 716.

The loader 712 can include a loader body 720 that can be sized to fit into the chamber 714. For example, the loader body 720 can be a generally cylindrical body. The loader body 720 can extend from a loader base 722 to a grip 724, with a grip direction being axially toward a terminus of the grip 724 and a base direction being axially toward the loader base 722. The loader base 722 can be a circular flange extending radially out from a base end of the loader body 720. Thus, the loader base 722 can inhibit insertion of the loader 712 into the chamber 714 with the loader base 722 leading because the loader base 722 can be designed and sized to inhibit insertion of the loader base 722 into the chamber 714. However, the loader 712 can be inserted into the chamber 714 with the grip end of the loader 712 being inserted first, followed by the loader body 720.

As an example, the grip 724 can include a pair of opposing jaws 726 that extend from the loader body 720 in a curved manner, like the jaws of the earplug loading apparatus 100 discussed above. The loader body 720 may be some other shape, such as where at least a portion of the loader body has a generally plus (+) shaped cross section or some other non-circular cross section instead of the circular cross section of a cylinder. The loader 712 can also have a catch 730, which can be a cantilever beam that extends from a wall of an aperture such as a cutout 732 defined in the loader body 720. The catch 730 and the cutout 732 can be like the catch and cutout of the loader 112 from the earplug loading apparatus 100 discussed above.

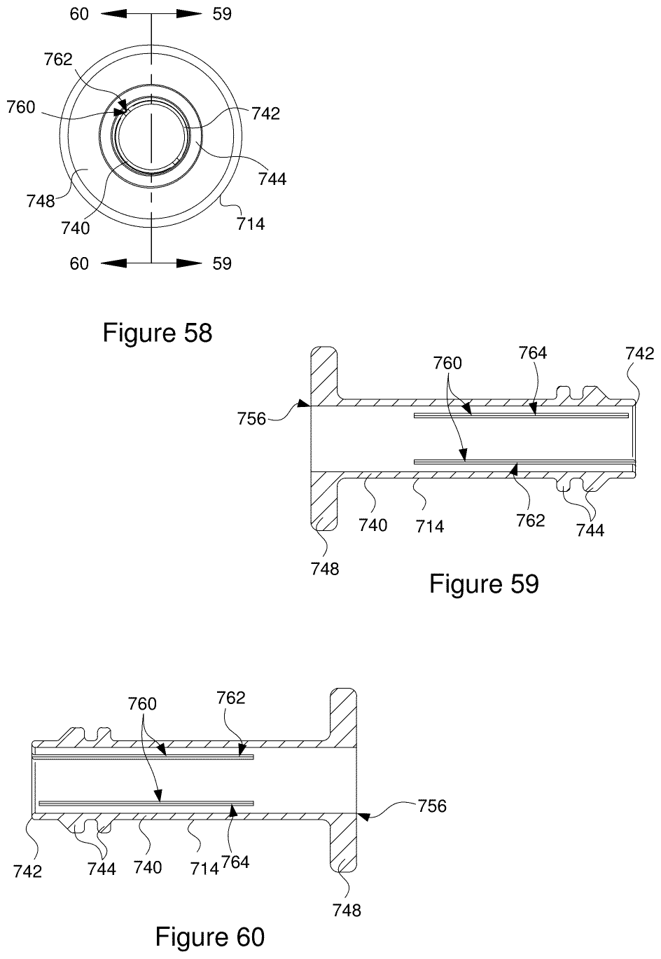

Referring now to FIGS. 56-60, the chamber 714 will be discussed in more detail. The chamber 714 can be like the chamber 114 discussed above, including having a chamber body 740, a distal tip 742, tip flanges 744, and a base flange 748 like the chamber 714. The funnel can be omitted, but a proximal face of the base flange 748 can form a catch area 756 for engaging the catch 730 to inhibit further insertion of the loader 712 into the chamber 714 after the catch 730 engages the catch area 756. However, a user can depress the catch 730 into the cutout 732 to allow the catch 730 to avoid engaging the catch area 756 and to be inserted into the chamber 714, allowing further insertion of the loader 712 into the chamber 714, such as when the loader 712 is to be used for loading an earplug 710 into the chamber 714.

The chamber body 740 can define axially extending slots 760 in the internal surfaces of the chamber body 740. The slots 760 may extend partially through the chamber body 740, or all or part of the slots 760 may extend entirely through the chamber body 740. The slots 760 can include a pair of distally open slots 762 and a pair of distally closed slots 764. The distally open slots 762 can be spaced apart circumferentially from each other, such as one-hundred and eighty degrees apart from each other. The distally open slots 762 can each extend in the proximal direction from the distal tip 742 of the chamber body 740 so that the distally open slots are open at their distal ends. The distally open slots 762 can extend proximally until they end at rear slot walls, which are located in the same proximal end plane that is perpendicular to a central axis of the chamber 714. The distally closed slots 764 can be spaced apart circumferentially from each other, such as one-hundred and eighty degrees apart from each other. The distally closed slots 764 can both extend proximally from the same distal end plane that is perpendicular to the central axis of the chamber 714. The distally closed slots 764 can each have a proximal end at the same proximal end plane as the distally open slots 762. Thus, the distally closed slots 764 can each have a proximal end wall and a distal end wall, while the distally open slots 762 can each have a proximal end wall but not a distal end wall (so that the distally open slots 762 are open on their distal ends). In an alternative design, the distally closed slots 764 may not have proximal end walls because the distally closed slots 764 may extend proximally all the way through the proximal end of the chamber 714 (so that the distally closed slots 764 are open on their proximal ends).

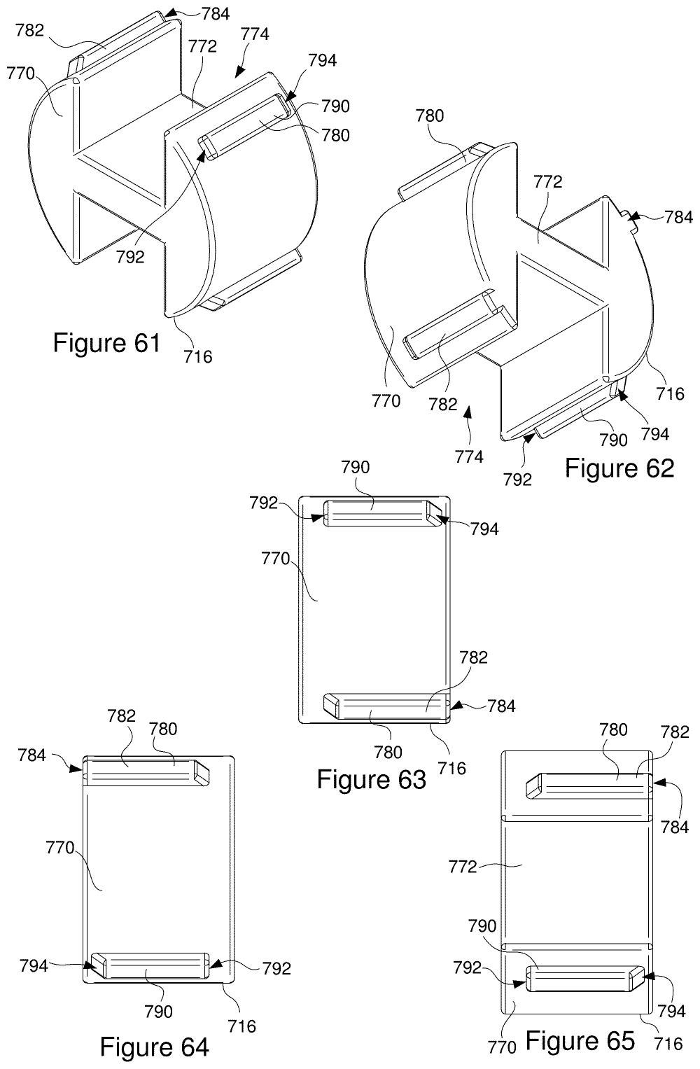

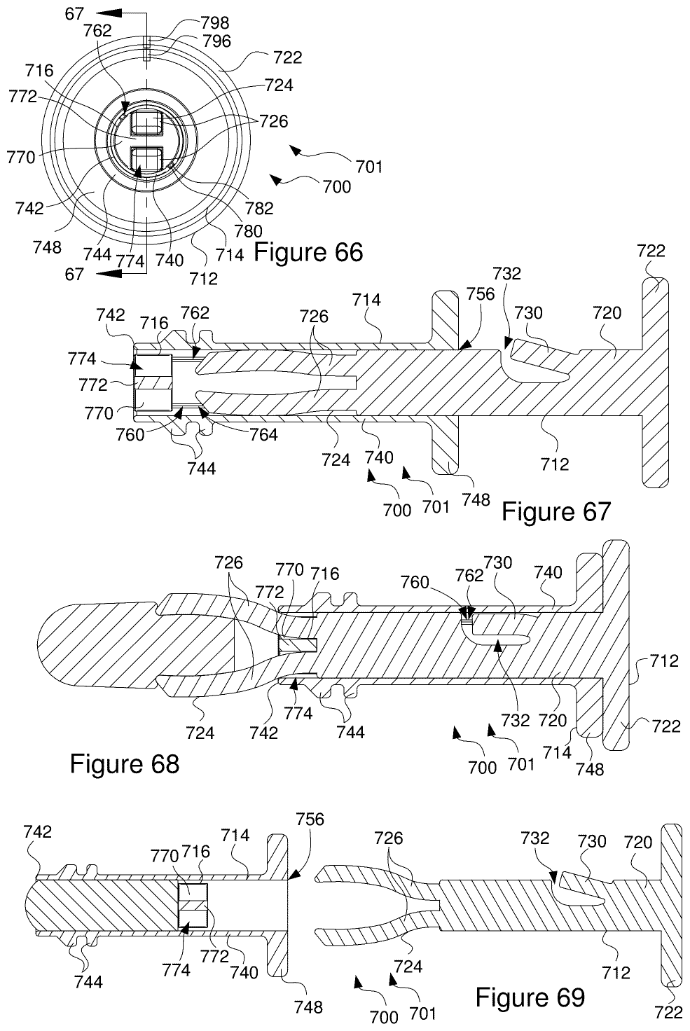

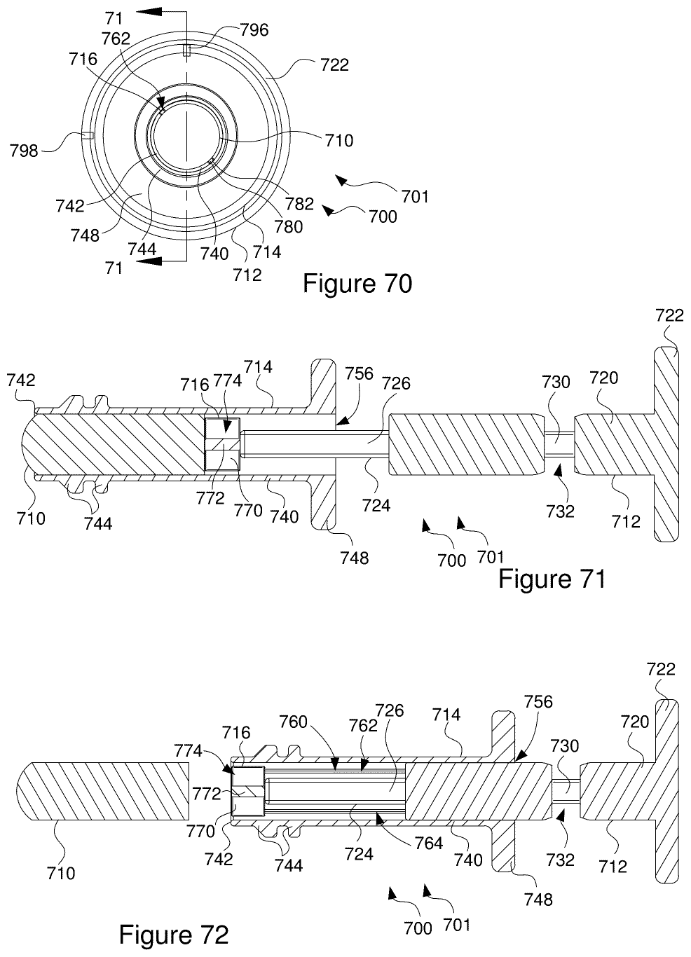

Referring now to FIGS. 61-65 and still to FIG. 55, the stop 716 will be discussed in more detail. The stop 716 can generally have a cross section that is in the shape of two minor segments defined by non-diametric parallel chords that can be parallel to each other, in addition to a rectangular area extending between the segments. A line that splits the rectangular area into two equal halves can extend through midpoints of the parallel chords. Thus, the stop 716 can include a pair of opposite minor segment areas 770 formed by the minor segments of the cross section. The stop 716 can also include a joining wall 772 joining the minor segment areas 770. Thus, the stop can define a pair of access gaps 774 between the minor segment areas 770 on both sides of the joining wall 772. The diameter of the circle defining the cross section of the minor segment areas 770 can be sized relative to an inner diameter of the chamber body 740 so that the minor segment areas 770 can slide inside the chamber body 740. Also, the access gaps 774 can be sized to receive the jaws 726 of the grip 724 of the loader 712, so that the jaws 726 can slide through the access gaps 774 if the loader 712 is rotated relative to the chamber 714 and the stop 716 so that the jaws 726 align with the access gaps 774 in a loading orientation illustrated in FIGS. 66-69. However, when the stop 716 is in the chamber 714, engagement between the jaws 726 and the stop 716 can inhibit movement of the jaws 726 past the stop 716 when the jaws 726 are not rotationally aligned with the access gaps 774 in an insertion orientation illustrated in FIGS. 70-72.

The stop 716 can also include guides 780, which can each extend radially out from the outer diameters of the minor segment areas 770 of the stop 716. The guides 780 can include a pair of proximal stop guides 782 that can be aligned with the distally open slots 762. The proximal stop guides 782 can be sized to slide into and along the distally open slots 762 as the stop 716 slides axially within the chamber 714. The proximal stop guides 782 can include proximal stop surfaces 784 on proximal ends of the proximal stop guides 782, which can engage proximal ends of the distally open slots 762 to inhibit further sliding movement in the proximal direction of the stop 716 relative to the chamber 714 beyond a position wherein the proximal stop surfaces 784 contact proximal end walls of the distally open slots 762. As an example, the proximal end walls of the distally open slots 762 and the proximal stop surfaces 784 of the proximal stop guides 782 may each include contact areas that are aligned with planes that are perpendicular to axes of the chamber 714 and of the minor segment areas 770 of the stop 716. As an alternative example, the proximal end walls of the distally open slots 762 and the proximal stop surfaces 784 of the proximal stop guides 782 may each include contact areas that hook into each other (such as with the proximal end walls of the distally open slots 762 and the proximal stop surfaces 784 of the proximal stop guides 782 (or at least contact areas of them) each sloping in the proximal direction as they extend radially out).

The guides 780 can also include a pair of distal stop guides 790 that can be aligned with the distally closed slots 764. The distal stop guides 790 can be sized to slide into and along the distally closed slots 764 as the stop 716 slides axially within the chamber 714. The distal stop guides 790 can include distal stop surfaces 792 on distal ends of the distal stop guides 790, which can engage distal ends of the distally closed slots 764 to inhibit further sliding movement in the distal direction of the stop 716 relative to the chamber 714 beyond a position wherein the distal stop surfaces 792 contact distal end walls of the distally closed slots 764. As an example, the distal end walls of the distally closed slots 764 and the distal stop surfaces 792 of the distal stop guides 790 may each include contact areas that are aligned with planes that are perpendicular to axes of the chamber 714 and of the minor segment areas 770 of the stop 716. As an alternative example, the distal end walls of the distally closed slots 764 and the distal stop surfaces 792 of the distal stop guides 790 may each include contact areas that hook into each other (such as with the distal end walls of the distally closed slots 764 and the distal stop surfaces 792 of the distal stop guides 790 (or at least contact areas of them) each sloping in the distal direction as they extend radially out).

Proximal ends of the distal stop guides 790 can be sloped to form wedge surfaces 794, which can slope in a distal direction as they extend radially out from the minor segment areas 770 of the stop 716. The distal stop guides 790 may be shortened, with the distal stop surfaces 792 being located farther proximally on the stop 716 from where they are illustrated in the figures. The distally closed slots 764 of the chamber 714 can be shortened by the same amount as the distal stop guides 790, so that the distal ends of the distally closed slots 764 can be located proximally from where there are illustrated in the figures. In such an embodiment, the stop 716 can define cutouts around the distal stop guides 790 similar to the cutouts around the guides 642 of the grip base 640 that are illustrated in FIG. 51 and discussed above. Such cutouts can allow the distal stop guides 790 to bend radially inwardly when a radially inward force is applied to the distal stop guides 790.

The proximal end plane that is coplanar with the proximal ends of the distally open slots 762 can be located at an axial position to allow the stop 716 to be slid proximally far enough along the slots to allow the earplug 710 to be seated in the chamber 714 between the stop 716 and the distal tip 742 of the chamber 714.

Thus, in use, the stop 716 can slide within the chamber 714 between a proximal position wherein the proximal stop surfaces 784 of the proximal stop guides 782 contact the proximal end walls of the distally open slots 762 (See FIGS. 69 and 71) and a distal position wherein distal stop surfaces 792 of the distal stop guides 790 contact the distal end walls of the distally closed slots 764 (see FIGS. 67-68 and 72). But the stop 716 can be inhibited from sliding in the distal direction beyond the distal position or from sliding in the proximal direction beyond the proximal position.

In use for loading, the loader 712 can be aligned with the chamber 714 in the loading orientation with the jaws 726 of the loader 712 aligning with the access gaps 774 of the stop 716. As illustrated in FIGS. 66 and 70, the chamber 714 and/or the loader 712 may include one or more alignment indicators that indicate whether the loader 712 and the chamber 714 are rotated relative to each other in the loading orientation so that the jaws 726 of the loader 712 are aligned with the access gaps 774 of the stop 716. For example, a chamber alignment indicator 796 (illustrated only in FIG. 66 and FIG. 70) may be marked on the base flange 748 of the chamber 714 and a loader alignment indicator 798 (illustrated only in FIG. 66 and FIG. 70) may be marked on the loader base 722 of the loader 712 so that the indicators align when the loader 712 and the chamber 714 are in the loading orientation of FIG. 66 and the marks are misaligned when the loader 712 and the chamber 714 are in the inserting orientation of FIG. 70. The loader alignment indicator 798 and/or the chamber alignment indicator 796 may be printed, imprinted, raised, and/or cut out of the respective parts of the loader 712 and the chamber 714.

Referring now to FIGS. 66-69, use of the earplug loading apparatus 700 to load the earplug 710 into the chamber 714 will be discussed. For loading, the loader 712 and the chamber 714 can be oriented in the loading orientation illustrated in FIGS. 66-67. The loader 712 can be inserted into the chamber 714 with the grip 724 of the loader 712 leading into the chamber 714 as illustrated in FIG. 67. The loader 712 can continue to be inserted, sliding in the distal direction relative to the chamber 714. The catch 730 of the loader 712 can be depressed into the cutout 732 of the loader 712 while pushing the loader 712 until the catch 730 passes into the chamber 714. The loader 712 can continue to be inserted in the distal direction relative to the chamber 714 until the jaws 726 of the grip 724 of the loader 712 slide through the access gaps 774 of the stop 716, with the distal portions of the jaws 726 protruding out and beyond the distal tip 742 of the loader 712, as illustrated in FIG. 68. In this position, the minor segment areas 770 of the stop can be positioned on opposite sides of each jaw 726, and the wall 772 between the minor segment areas 770 can be positioned between the jaws 726. With the loader 712 and the chamber 714 in the position illustrated in FIG. 68, the earplug 710 can be inserted between the jaws 726 of the loader 712, as is also illustrated in FIG. 68.

With the jaws 726 of the loader gripping the earplug 710 as illustrated in FIG. 68, the loader 712 can be pulled in the proximal direction relative to the chamber 714. As this happens, the chamber 714 can squeeze the jaws 726 inwardly toward each other as the jaws are pulled into the chamber 714, tightening the grip of the jaws 726 on the earplug 710. Additionally, the jaws can pull the earplug into the chamber 714. This proximal direction movement can also slide the stop 716 in the proximal direction in the chamber 714 until movement of the stop 716 is stopped as the proximal stop surfaces 784 of the proximal stop guides 782 engage the proximal end walls of the distally open slots 762. The stop 716 can in turn inhibit further proximal movement of the earplug 710 along the chamber 714. However, the loader 712, including the jaws 726, can continue to slide proximally relative to the chamber until the loader 712 has been removed from the chamber 714 as illustrated in FIG. 69, with the earplug 710 still loaded in the chamber 714 in a position wherein a proximal end of the earplug 710 can be seated against a distal end of the stop 716. However, the loader 712 may remain partially inserted in the chamber 714.

Referring now to FIGS. 70-72, use of the earplug loading apparatus 700 to push or plunge the earplug 710 from the chamber 714 and insert the earplug 710 into an ear canal will be discussed. As illustrated in FIGS. 70-71, the loader 712 can be inserted into the chamber 714 with the jaws 726 of the loader being inserted first. The loader 712 can be in an orientation relative to the chamber 714 that is in the inserting position, such as a position that is rotated ninety degrees relative to the loading position. For example, in this inserting position, the chamber alignment indicator 796 and the loader alignment indicator 798 may be rotated ninety degrees apart from each other. In this inserting position, the stop 716 can engage the jaws 726 of the loader 712 to inhibit insertion of the jaws 726 through the stop 716. Thus, after the jaws 726 engage the stop 716, further insertion of the loader 712 into the chamber 714 can push the stop 716 distally along the chamber 714, and the stop 716 can push the earplug 710 distally along and out of the chamber 714. This motion can continue until the stop 716 reaches its distal position illustrated in FIG. 70 as it pushes the earplug 710 out of the chamber 714 and inserts the earplug 710 into an ear canal of a user (see FIG. 72). Thus, the loader 712 can engage the earplug 710 via the stop 716 to push and eject the earplug 710 from the chamber 714 via the stop 716. The engagement between the stop 716 and the chamber 714 can inhibit further movement of the stop 716 beyond the distal position of the stop 716, which can also inhibit further movement of the loader 712 in the distal direction beyond the position illustrated in FIG. 72.

The loader 712 can be used to load additional earplugs into the chamber 714 in the same manner described above for the earplug 710, and such earplugs can be plunged from the chamber and inserted into an ear canal in the same manner as described above for the earplug 710.

The components of the earplug loading apparatus 700 can be made of materials that are appropriate for the functions and structures described herein. For example, the loader 712, the stop 716, and the chamber 714 may be made of polymer materials, composite materials, and/or metals. For example, the materials may be appropriately strong, stiff/flexible, and durable. The loader 712, the stop 716, and/or the chamber 714 can be formed with manufacturing techniques such as molding, milling, lathing, and/or additive manufacturing such as 3D printing. As with other earplugs discussed herein, the earplug 710 can be made of a compliant material such as foam, and they can be formed by manufacturing processes such as molding.

To assemble the stop 716 in the chamber 714, the stop 716 can be oriented so that the guides 780 are aligned with the slots 760, and specifically with the proximal stop guides 782 being aligned with the distally open slots 762 and the distal stop guides 790 being aligned with the distally closed slots 764. Also, the wedge surfaces 794 of the distal stop guides 790 can be positioned closest to the distal tip 742 of the chamber 714, which can be the same relative orientation of the stop 716 and the chamber 714 illustrated in FIG. 55 and other figures in the drawings. In this orientation, the stop 716 can be pushed into the distal end of the chamber 714. The sloped wedge surfaces 794 can act as wedges to push the adjacent portions of the chamber 714 outward and/or push the distal stop guides 790 radially inward (especially if cutouts are defined around the distal stop guides as discussed above), allowing the distal stop guides 790 to pass axially into the distally closed slots 764. The proximal stop guides 782 can also slide into the distally open slots 762 without needing such wedge action because the distally open slots 762 can be open on their distal ends. With the guides 780 positioned in the slots 760, the adjacent portions of the chamber 714 (those that had been pushed outward by the distal stop guides 790) can spring back inward, inhibiting movement of the stop 716 back out of the chamber 714. Alternatively, the components of the chamber 714 and the stop 716 can be formed with the stop 716 already positioned in the chamber 714 using additive manufacturing techniques such as 3D printing. Also, for any of the components in any of the embodiments discussed herein, the components can be shaped differently to achieve the functions of such components, considering implementation details such as the materials to be used for such components.

V. Additional Earplug Loading Apparatus Example with Expanded Chamber

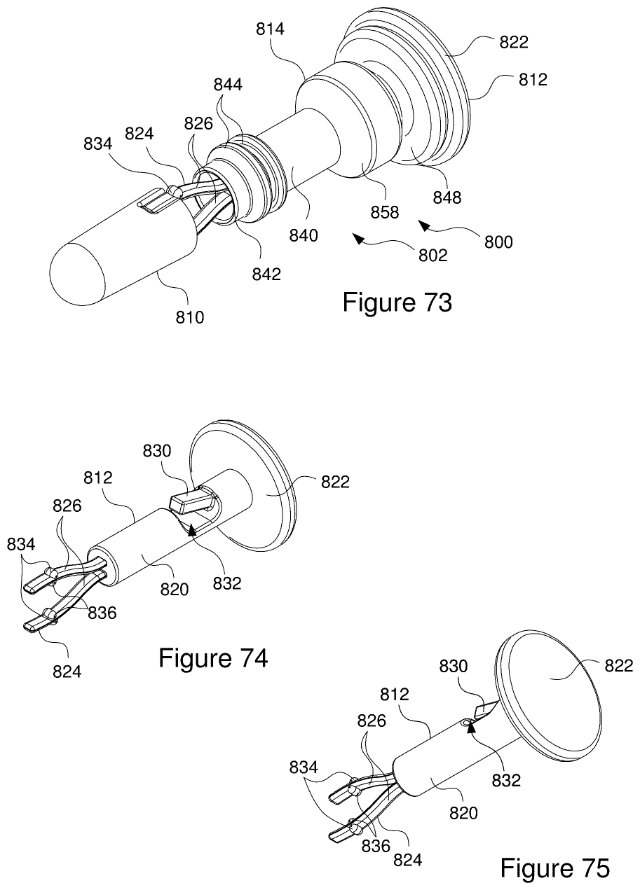

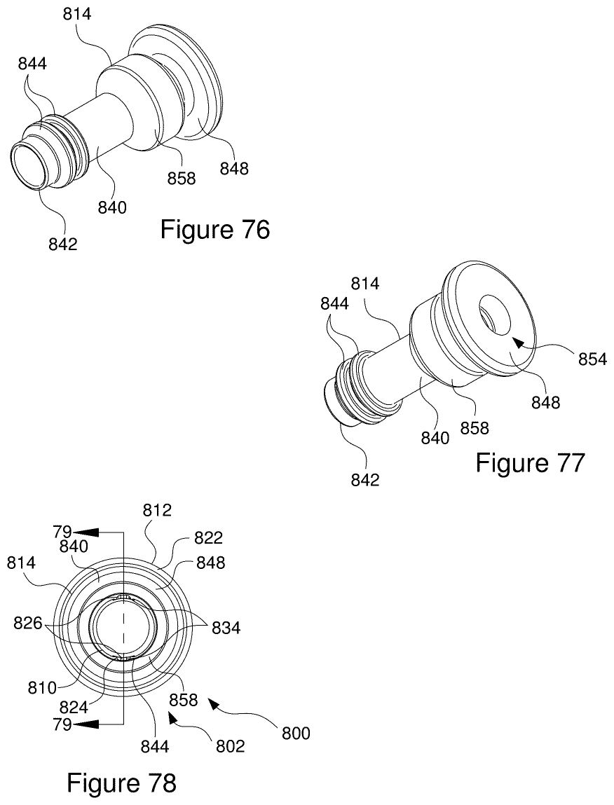

Referring to FIGS. 73-81, another example of an earplug loading apparatus 800 with an earplug insertion device 101 will be discussed. The earplug loading apparatus 800 can be used to load an earplug 810 like the earplugs discussed above and insert the earplug into an ear canal 106, as will be discussed in more detail below.

The earplug loading apparatus can include a loader 812 and a chamber 814. The loader 812 can be like the loader 712 discussed above, including a loader body 820, a loader base 822, a grip 824 with opposing jaws 826, a catch 830, and a cutout 832 defined in the loader body 820. However, each of the jaws 826 may be shaped differently from the jaws 826, including each jaw 826 including an outwardly protruding nub 834, which can protrude outwardly from an outer area of the curved jaw 826 in an outward direction away from the other jaw 826. Each of the jaws 826 may also include an inwardly protruding nub 836, which can protrude inwardly from the outer area of the curved jaw 826 in an inward direction toward the other jaw 826. Each inwardly protruding nub 836 may be located near the outwardly protruding nub 834 on the same jaw 826. For example, each inwardly protruding nub 836 may be axially located farther in the distal direction along the jaw 826 than the corresponding outwardly protruding nub 834, or at about the same axial location as the corresponding outwardly protruding nub 834.

The chamber 814 can be like the chamber 114 discussed above, including a chamber body 840, a distal tip 842, distal tip flanges 844, a base flange 848, and a catch area 854 on a proximal side of the base flange 848 (see FIG. 77). The chamber 814 can omit the funnel 154. Also, the chamber 814 can include an expanded section 858 between the distal tip flanges 844 and the base flange 848. For example, the expanded section 858 can have inner and outer surfaces that start at a distal end of the expanded section, extending radially out as they extend in the proximal direction in a distal frustoconical portion, then extending axially in a cylindrical portion, and then extending radially in as they extend in the proximal direction in a proximal frustoconical portion. As is discussed below, the expanded section 858 can allow the jaws 826 of the loader 812 to expand out when the jaws 826 are at least partially within the expanded section 858 to loosen the grip of the jaws 826 on the earplug 810.

Referring to FIGS. 78-81, use of the earplug loading apparatus 800 will be discussed. As illustrated in FIG. 73 and FIGS. 78-79, the loader 812 can be positioned through the chamber 814, with the jaws 826 of the loader 812 protruding out of the distal tip 842 of the chamber 814, with the flange of the loader base 822 abutting the catch area 854 of the chamber 814. The earplug 810 can be placed between the jaws 826 so that the jaws 826 grip the earplug 810, as is also illustrated in FIGS. 73 and 78-79. A proximal end of the earplug 810 may be located at the inwardly protruding nubs 836, with the inwardly protruding nubs 836 helping to locate the earplug 810 and indicate an axial location where the proximal end of the earplug 810 is to be placed. Thus, the proximal end of the earplug 810 may be placed axially at the outwardly protruding nubs 834 or farther in the distal direction from the outwardly protruding nubs 834 (but possibly with some portion of the earplug 810 still extending proximally past the inwardly protruding nubs 836). The loader 812 can be pulled in the proximal direction relative to the chamber 814 to pull the earplug 810 into the chamber 814. As the jaws 826 are pulled into the chamber 814, the chamber 814 can squeeze the jaws 826 toward each other, which can cause the jaws 826 to grip the earplug 810 more tightly as the jaws 826 enter the chamber 814. The outwardly protruding nubs 834 of the jaws 826 can contact the chamber 814 as the outwardly protruding nubs 834 are pulled into the chamber 814 to cause the jaws 826 to press further toward each other and grip the earplug 810 more tightly as the outwardly protruding nubs 834 enter the chamber 814. As the outwardly protruding nubs 834 enter the expanded section 858 of the chamber 814, the outwardly protruding nubs 834 and the remainder of the jaws 826 can expand away from each other, opening and loosening the grip of the jaws 826 on the earplug 810 until the grip of the jaws 826 is loose enough to release the earplug 810, leaving the earplug 810 loaded in the chamber 814 as the loader 812 continues moving in the proximal direction relative to the chamber 814. For example, the earplug 810 might be released so that the earplug 810 is located in the narrow part of the chamber 814 and does not extend proximally into the expanded section 858 of the chamber 814, or so that only a small portion of the earplug 810 extends into the expanded section 858 of the chamber 814.

Beginning in the position illustrated in FIG. 80 with the earplug 810 loaded in the chamber 814 and the tips of the jaws 826 of the loader 812 being positioned near or against the proximal end of the earplug 810, the earplug loading apparatus 800 can operate as an earplug insertion device 802. The distal tip 842 of the chamber 814 can be placed in the opening of an ear canal, and the loader 812 can be pushed in the distal direction relative to the chamber 814 until the catch 830 contacts or catches on the catch area 854, pushing the earplug 810 out of the chamber 814 and into the ear canal, as illustrated in FIG. 81. As illustrated in FIG. 81, the jaws 826 may not quite reach the extreme distal tip 842 of the chamber 814, but they may still reach far enough to expel the earplug 810 from the chamber 814 and into the ear canal. Also, the jaws 826 may be longer than illustrated in the figures, so that the jaws 826 are sufficiently long to expel the earplug 810 from the chamber 814 and into the ear canal. The earplug loading and insertion operations discussed here can be repeated with different earplugs and/or the same earplugs to load earplugs into the chamber 814 and insert the earplugs into the same ear canal and/or different ear canals.

The components of the earplug loading apparatus 800 can be made of materials that are appropriate for the functions and structures described herein. For example, the loader 812 and the chamber 814 may be made of polymer materials, composite materials, and/or metals. For example, the materials may be appropriately strong, stiff/flexible, and durable. The loader 812 and/or the chamber 814 can be formed with manufacturing techniques such as molding, milling, lathing, and/or additive manufacturing such as 3D printing. As with other earplugs discussed herein, the earplug 810 can be made of a compliant material such as foam, and the earplug can be formed by manufacturing processes such as molding.

VI. Another Earplug Loading Apparatus Example with Stop

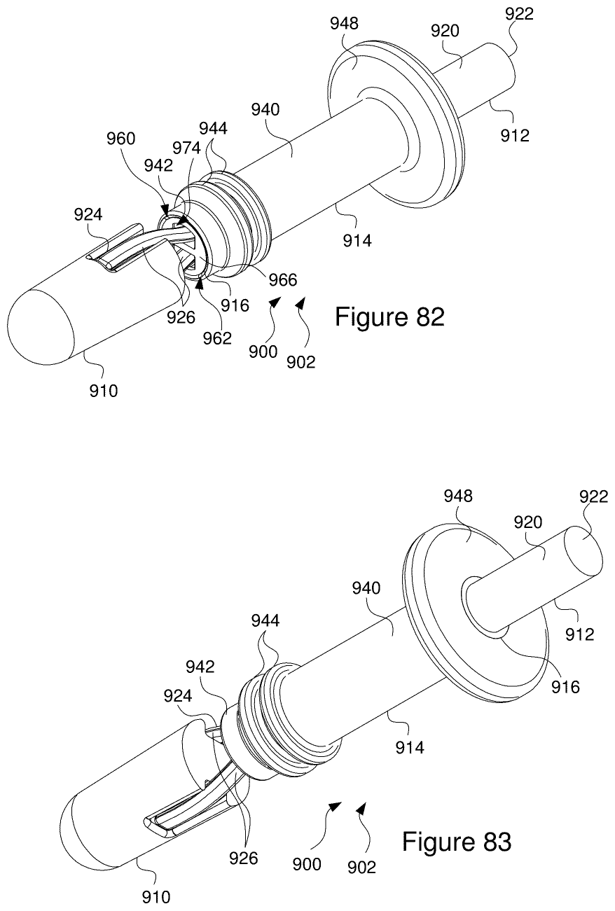

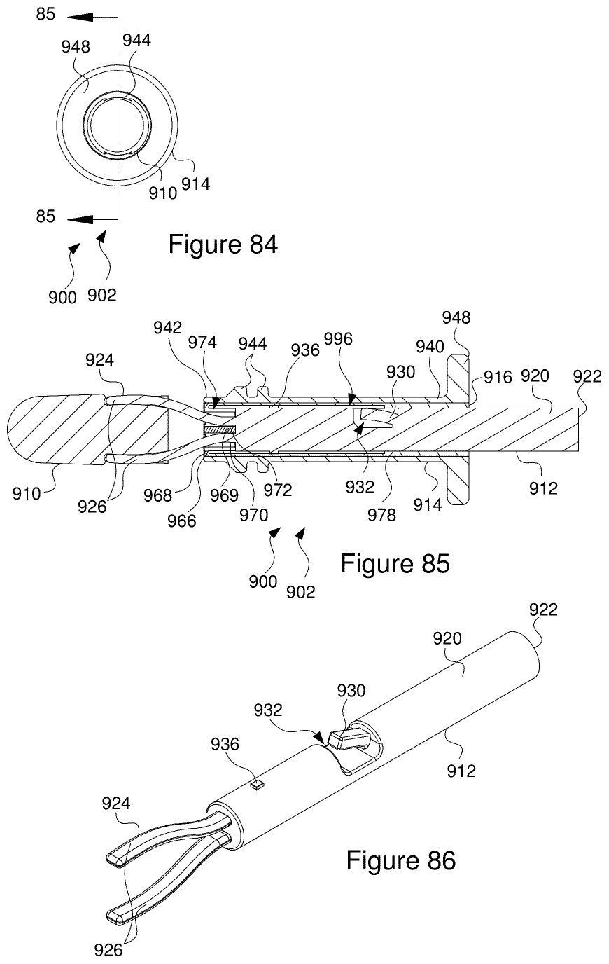

Referring to FIGS. 82-98, another earplug loading apparatus 900, which can include an earplug insertion device 902, will be discussed. The earplug loading apparatus 900 can include an earplug 910, a loader 912, a chamber 914, and a stop 916.

The loader 912 can be structured to load the earplug 910 (and/or some other earplug) into the chamber 914 (i.e., at least partially in the chamber 914, and possibly fully in the chamber 914). The loader 912 can also be structured to push the earplug 910 out of the chamber 914 and into the ear canal of the user (like the ear canal 106 of the user 102 discussed above) via the stop 916.

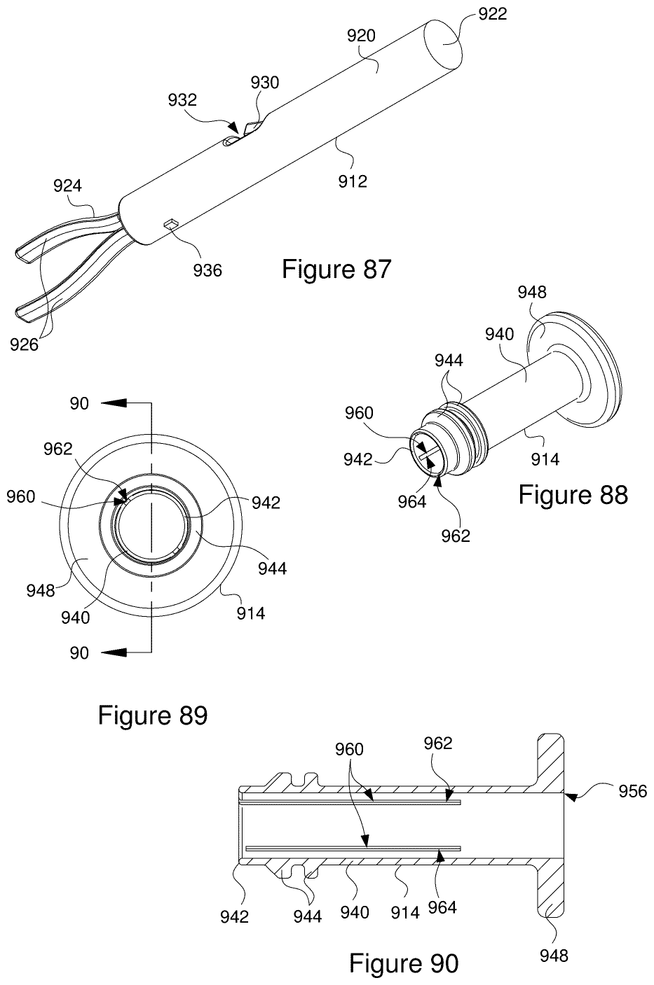

As illustrated in FIGS. 86-87, the loader 912 can include a loader body 920 that can be sized to fit into the stop 916, which can be sized to fit into the chamber 914. For example, the loader body 920 can be a generally cylindrical body. The loader body 920 can extend from a loader base 922 to a grip 924, with a grip direction (which is also a distal direction in this example because the loader 912 can be oriented the same way relative to the chamber 914 for loading and inserting operations) being axially toward a terminus of the grip 924 and a base direction (which is also a proximal direction in this example) being axially toward the loader base 922.

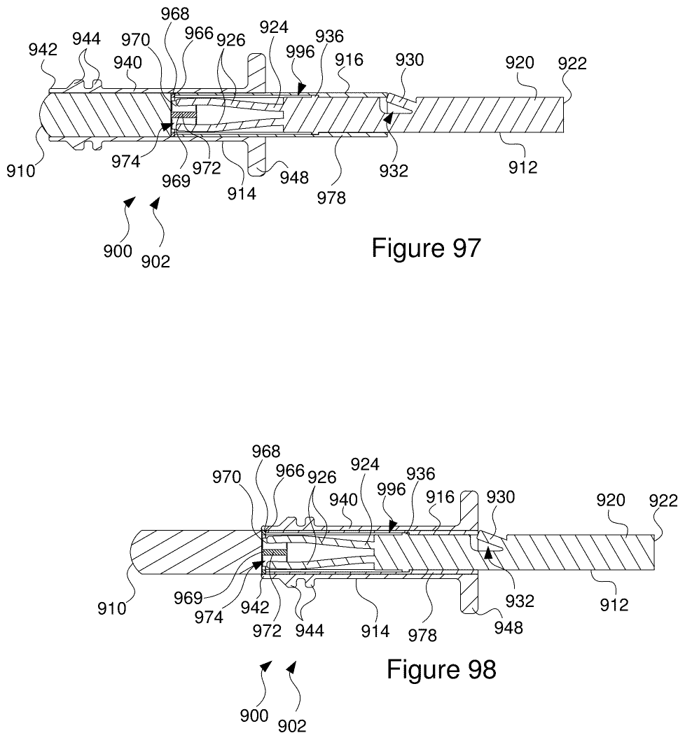

As an example, the grip 924 can include a pair of opposing jaws 926 that extend from the loader body 920 in a curved manner, like the jaws of the loader 512 discussed above. As with the other loader bodies discussed herein, the loader body 920 may be some other shape, such as where at least a portion of the loader body has a generally plus (+) shaped cross section or some other non-circular cross section instead of the circular cross section of a cylinder. The loader 912 can also have a catch 930, which can be a cantilever beam that extends from a wall of an aperture such as a cutout 932 defined in the loader body 920. The catch 930 and the cutout 932 can be like the catch and cutout of the loader 112 from the earplug loading apparatus 100 discussed above, but the catch 930 can be structured to contact a proximal end of the stop 916 when the catch 930 is extended (see FIGS. 97-98) and to fit within the stop 916 when the catch 930 is depressed (see FIG. 85).

The loader 912 can also include at least one guide, such as two guides 936 that protrude radially out from opposite sides of the loader body 920. For example, the guides 936 may be located axially between the grip 924 and the catch 930.

Referring to FIGS. 89-90, the chamber 914 can be like the chamber 714 illustrated in FIGS. 56-60 and described above. Like the chamber 714, the chamber 914 can include a chamber body 940, a distal tip 942, distal tip flanges 944, a base flange 948, a catch area 956 (see FIG. 90), and axially extending slots 960 along its inner surface that can include distally open slots 962 and distally closed slots 964.

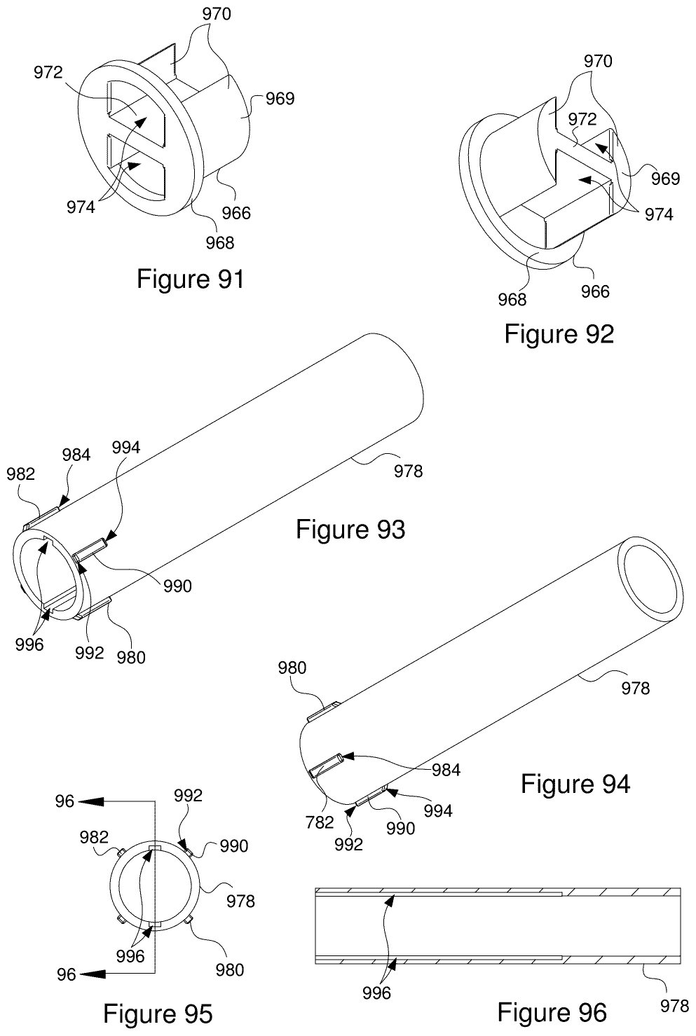

Referring to FIGS. 91-92, the stop 916 can include a stop cap 966, which can include a distal flange 968 extending out in a ring from a distal end of a cap body 969. The cap body 969 can extend in the proximal direction from the distal flange 968. The cap body 969 can generally have a cross section that is in the shape of two minor segments defined by non-diametric parallel chords that can be parallel to each other, in addition to a rectangular area extending between the segments. A line that splits the rectangular area into two equal halves can extend through midpoints of the parallel chords. Thus, the cap body 969 of the stop 916 can include a pair of opposite minor segment areas 970 formed by the minor segments of the cross section. The cap body 969 can also include a joining wall 972 joining the minor segment areas 970. Thus, the cap body 969 can define a pair of access gaps 974 between the minor segment areas 970 on both sides of the joining wall 972, and extending between the distal flange 968 (the portion of the distal flange that extends around the access gaps 974 between the minor segment areas 970) and the joining wall 972. The diameter of a circle defining the outer diameter of the cross section of the minor segment areas 970 can be sized relative to an inner diameter of a stop sleeve 978 of the stop 916 so that the minor segment areas 970 can slide inside the stop sleeve 978. Also, the access gaps 974 can be sized to receive the jaws 926 of the grip 924 of the loader 912, so that the jaws 926 can slide through the access gaps 974.

Referring to FIGS. 93-96, the stop sleeve 978 can include a cylindrical tube body. The stop sleeve 978 can also include guides 980 extending out from the stop sleeve 978 near a distal end of the stop sleeve 978.

The guides 980 can be like the guides 780 on the stop 716 discussed above. Thus, the guides 980 can include a pair of proximal stop guides 982 that can be aligned with the distally open slots 962. The proximal stop guides 982 can be sized to slide into and along the distally open slots 962 as the stop 916 slides axially within the chamber 914. The proximal stop guides 982 can include proximal stop surfaces 984 on proximal ends of the proximal stop guides 982, which can engage proximal ends of the distally open slots 962 to inhibit further sliding movement in the proximal direction of the stop 916 relative to the chamber 914 beyond a position wherein the proximal stop surfaces 984 contact proximal end walls of the distally open slots 962. As an example, the proximal end walls of the distally open slots 962 and the proximal stop surfaces 984 of the proximal stop guides 982 may each include contact areas that are aligned with planes that are perpendicular to axes of the chamber 914 and of the minor segment areas 970 of the stop 916. As an alternative example, the proximal end walls of the distally open slots 962 and the proximal stop surfaces 984 of the proximal stop guides 982 may each include contact areas that hook into each other (such as with the proximal end walls of the distally open slots 962 and the proximal stop surfaces 984 of the proximal stop guides 982 (or at least contact areas of them) each sloping in the proximal direction as they extend radially out).

The guides 980 can also include a pair of distal stop guides 990 that can be aligned with the distally closed slots 964. The distal stop guides 990 can be sized to slide into and along the distally closed slots 964 as the stop 916 slides axially within the chamber 914. The distal stop guides 990 can include distal stop surfaces 992 on distal ends of the distal stop guides 990, which can engage distal ends of the distally closed slots 964 to inhibit further sliding movement in the distal direction of the stop 916 relative to the chamber 914 beyond a position wherein the distal stop surfaces 992 contact distal end walls of the distally closed slots 964. As an example, the distal end walls of the distally closed slots 964 and the distal stop surfaces 992 of the distal stop guides 990 may each include contact areas that are aligned with planes that are perpendicular to axes of the chamber 914 and of the minor segment areas 970 of the stop 916. As an alternative example, the distal end walls of the distally closed slots 964 and the distal stop surfaces 992 of the distal stop guides 990 may each include contact areas that hook into each other (such as with the distal end walls of the distally closed slots 964 and the distal stop surfaces 992 of the distal stop guides 990 (or at least contact areas of them) each sloping in the distal direction as they extend radially out).

Proximal ends of the distal stop guides 990 can be sloped to form wedge surfaces 994, which can slope in a distal direction as they extend radially out from the minor segment areas 970 of the stop 916. The distal stop guides 990 may be shortened, with the distal stop surfaces 992 being located farther proximally on the stop 916 from where they are illustrated in the figures. The distally closed slots 964 of the chamber 914 can be shortened by the same amount as the distal stop guides 990, so that the distal ends of the distally closed slots 964 can be located proximally from where they are illustrated in the figures. In such an embodiment, the stop 916 can define cutouts around the distal stop guides 990 similar to the cutouts around the guides 642 of the grip base 640 that are illustrated in FIG. 51 and discussed above. Such cutouts can allow the distal stop guides 990 to bend radially inwardly when a radially inward force is applied to the distal stop guides 990.

The proximal end plane that is coplanar with the proximal ends of the distally open slots 962 can be located at an axial position to allow the stop 916 to be slid proximally far enough along the slots to allow the earplug 910 to be seated in the chamber 914 between the stop 916 and the distal tip 942 of the chamber 914.

Thus, in use, the stop 916 can slide within the chamber 914 between a proximal position wherein the proximal stop surfaces 984 of the proximal stop guides 982 contact the proximal end walls of the distally open slots 962 (See FIG. 97) and a distal position wherein distal stop surfaces 992 of the distal stop guides 990 contact the distal end walls of the distally closed slots 964 (see FIGS. 82-85 and 98). But the stop 916 can be inhibited from sliding in the distal direction beyond the distal position or from sliding in the proximal direction beyond the proximal position.

The stop sleeve 978 can also define at least one distally open slot 996 extending axially in the proximal direction from the distal end of the stop sleeve 978 and extending radially out from an inner surface of the stop sleeve 978. The distally open slots 996 can be sized and positioned to receive the guides 936 of the loader 912. The stop cap 966 can be joined with the stop sleeve 978 to form the stop 916, with the distal flange 968 of the stop cap 966 abutting a distal end of the stop sleeve 978 and with the cap body 969 of the stop cap 966 extending in the proximal direction into the stop sleeve 978. As an example, the stop cap 966 can be joined to the cap body 969 with an adhesive.

The loader 912 can be positioned at least partially in the stop 916, with the guides 936 of the loader 912 sliding along the slots 996 as the loader 912 moves relative to the stop 916. However, proximal direction movement of the guides 936 of the loader 912 in the slots 996 can be stopped by engagement between the guides 936 and the proximal ends of the slots 996. In that proximal position of the loader 912 relative to the stop 916 illustrated in FIGS. 97-98, the jaws 926 of the loader 912 can be retracted into the stop 916. Additionally, in that proximal position, the catch 930 can be extended to engage the proximal end of the stop sleeve 978, thus inhibiting distal direction movement of the loader 912 relative to the stop 916. Accordingly, in this proximal position, distal direction movement and proximal direction movement of the loader 912 relative to the stop 916 can be inhibited. However, the catch 930 can be depressed to the retracted position illustrated in FIG. 85 to allow the catch 930 to slide in the distal direction into the stop sleeve 978, as illustrated in FIG. 85. When the loader 912 is pulled in the proximal direction to the proximal position relative to the stop 916, the catch 930 can spring back out to its extended position to again inhibit distal direction movement of the loader 912 relative to the stop 916. Additionally, distal direction movement of the loader 912 relative to the stop 916 can be stopped by engagement between a distal end of the loader body 920 and a proximal end of the cap body 969 of the stop cap 966.

Referring now to FIGS. 82-85 and FIGS. 97-98, use of the earplug loading apparatus 900 to load the earplug 910 into the chamber 914 will be discussed. For loading, the loader 912 and the chamber 914 can be configured in the loading configuration of FIGS. 82-85, with the loader 912 and the stop 916 both in their distal positions relative to the chamber 914. If the loader 912 and the stop 916 had been in their proximal positions as illustrated in FIG. 97, then to reach the distal positions, the loader 912 and the stop 916 could be pushed in the distal direction relative to the chamber 914 to push the stop 916 in the distal direction relative to the chamber 914. The stop can stop in the distal position illustrated in FIG. 98. The catch 930 could be depressed radially into the retracted position illustrated in FIG. 85, and the loader 912 could be pushed in the distal direction relative to the chamber 914 and the stop 916. The loader 912 could continue to be inserted in the distal direction relative to the chamber 914 until the jaws 926 of the grip 924 of the loader 912 slid through the access gaps 974 of the stop 916, with the distal portions of the jaws 926 protruding out and beyond the distal tip 942 of the chamber 914, as illustrated in FIGS. 82-85. In this position, the minor segment areas 970 of the stop 916 can be positioned on opposite sides of each jaw 926, and the wall 972 between the minor segment areas 970 can be positioned between the jaws 926. With the loader 912 and the chamber 914 in the position illustrated in FIGS. 82-85, the earplug 910 can be inserted between the jaws 926 of the loader 912, as is also illustrated in FIGS. 82-85.

With the jaws 926 of the loader gripping the earplug 910 as illustrated in FIGS. 82-85, the loader 912 can be pulled in the proximal direction relative to the chamber 914. As this happens, the chamber 914 can squeeze the jaws 926 inwardly toward each other as the jaws are pulled into the chamber 914, tightening the grip of the jaws 926 on the earplug 910. Additionally, the jaws 926 can pull the earplug 910 into the chamber 914. This proximal direction movement can also slide the stop 916 in the proximal direction in the chamber 914 until movement of the stop 916 is stopped as the proximal stop surfaces 984 of the proximal stop guides 982 engage the proximal end walls of the distally open slots 962 of the chamber 914. The stop 916 can in turn inhibit further proximal movement of the earplug 910 along the chamber 914. However, the loader 912, including the jaws 926, can continue to slide proximally relative to the chamber until the loader 912 reaches its proximal position relative to the stop 916 as illustrated in FIG. 97, with the earplug 910 still loaded in the chamber 914 in a position wherein a proximal end of the earplug 910 can be seated against a distal end of the stop 916.

Referring now to FIGS. 97-98, use of the earplug loading apparatus 900 to push or plunge the earplug 910 from the chamber 914 and insert the earplug 910 into an ear canal will be discussed. As illustrated in FIG. 97, the loader 912 and the stop 916 can each be in their proximal positions (with the stop 916 in its proximal position relative to the chamber 914 and the loader 912 in its proximal position relative to the stop 916). From there, with the distal tip 942 of the chamber 914 positioned in an opening of an ear canal, the loader 912 and/or the stop 916 can be pushed in the distal direction. If the loader 912 is pushed, the catch 930 of the loader 912 can push the proximal end of the stop 916 in the distal direction along with the loader 912. As this happens, the stop 916 can push the earplug 910 in the distal direction relative to the chamber 914. Thus, the loader 912 can be used to push the earplug in the distal direction relative to the chamber 914 via the stop 916. This motion can continue until the stop 916 reaches its distal position illustrated in FIG. 98 as it pushes the earplug 910 out of the chamber 914 and inserts the earplug 910 into an ear canal of a user (see FIG. 98, with the ear canal not being illustrated). Thus, the loader 912 can engage the earplug 910 via the stop 916 to push and eject the earplug 910 from the chamber 914 via the stop 916. The engagement between the stop 916 and the chamber 914 can inhibit further movement of the stop 916 beyond the distal position of the stop 916, which can also inhibit further movement of the loader 912 in the distal direction beyond the position illustrated in FIG. 98 due to the catch 930 of the loader 912 engaging the stop 916.

The loader 912 can be used to load additional earplugs into the chamber 914 in the same manner described above for the earplug 910, and such earplugs can be plunged from the chamber and inserted into an ear canal in the same manner as described above for the earplug 910.

The components of the earplug loading apparatus 900 can be made of materials that are appropriate for the functions and structures described herein. For example, the loader 912, the stop 916, and the chamber 914 may be made of polymer materials, composite materials, and/or metals. For example, the materials may be appropriately strong, stiff/flexible, and durable. The loader 912, components of the stop 916, and/or the chamber 914 can be formed with manufacturing techniques such as molding, milling, lathing, and/or additive manufacturing such as 3D printing. As with other earplugs discussed herein, the earplug 910 can be made of a compliant material such as foam, and they can be formed by manufacturing processes such as molding.

To assemble the earplug loading apparatus 900, the stop sleeve 978 can be inserted in the chamber 914. Specifically, the stop sleeve 978 can be oriented so that the guides 980 are aligned with the slots 960, and specifically with the proximal stop guides 982 being aligned with the distally open slots 962 and the distal stop guides 990 being aligned with the distally closed slots 964. Also, the wedge surfaces 994 of the distal stop guides 990 can be positioned so they are facing toward the distal tip 942 of the chamber 914, which can be the same relative orientation of the stop 916 and the chamber 914 illustrated in FIGS. 82-85 and FIGS. 97-98. In this orientation, the stop sleeve 978 can be pushed into the distal end of the chamber 914. The sloped wedge surfaces 994 can act as wedges to push the adjacent portions of the chamber 914 outward and/or push the distal stop guides 990 radially inward (especially if cutouts are defined around the distal stop guides as discussed above), allowing the distal stop guides 990 to pass axially into the distally closed slots 964. The proximal stop guides 982 can also slide into the distally open slots 962 without needing such wedge action because the distally open slots 962 can be open on their distal ends. With the guides 980 positioned in the slots 960, the adjacent portions of the chamber 914 (those that had been pushed outward by the distal stop guides 990) can spring back inward and/or the distal stop guides 990 can spring back outward, inhibiting movement of the stop 916 back out of the chamber 914.

Changes could be made to the example earplug loading apparatus 900 and to the other earplug loading apparatus examples discussed herein. For example, as with other examples above, an earplug loading apparatus like the earplug loading apparatus 900 could include a loader with three or more jaws instead of two jaws, with the three or more jaws being sized and shaped like the jaws 926 or sized and shaped differently from the jaws 926 illustrated in the figures and discussed above. Also, the stop could include the same number of access gaps as the number of loader jaws (such as three or more access gaps where there are three or more jaws) or larger access gaps if multiple jaws can fit through a single access gap, so that the loader jaws could pass through the access gaps like the jaws 926 pass through the access gaps 974 discussed above.

The loader 912 can be inserted into the stop sleeve 978. Specifically, the loader 912 can be oriented as in the figures with the jaws 926 facing in the distal direction with the guides 936 of the loader 912 being circumferentially aligned with the slots 996 in the stop sleeve 978, as illustrated in the figures. Then, the loader base 922 can be inserted into the distal end of the stop sleeve 978, followed by the remainder of the loader 912. The catch 930 can be depressed by the stop sleeve 978 as the catch 930 enters the stop sleeve 978, and the guides 936 of the loader 912 can slide along the slots 996 of the stop sleeve 978. An adhesive can be placed on the stop cap 966 (on the surfaces that will contact the stop sleeve 978, such as on the outer surfaces of the cap body 969 and/or on the proximal side of the distal flange 968) and the cap body 969 can be oriented with the access gaps 974 aligning with the jaws 926 of the loader 912 and with the cap body 969 facing in the proximal direction. The cap body 969 can then be inserted in the distal end of the stop sleeve 978 to form the stop 916, which can include the stop cap 966 and the stop sleeve 978. The adhesive can be allowed to dry to secure the stop cap 966 in place in the stop sleeve 978.

Alternatively, some or all of the components of the earplug loading apparatus 900 can be formed already assembled using additive manufacturing techniques such as 3D printing.

VII. Yet Another Earplug Loading Apparatus Example with Stop

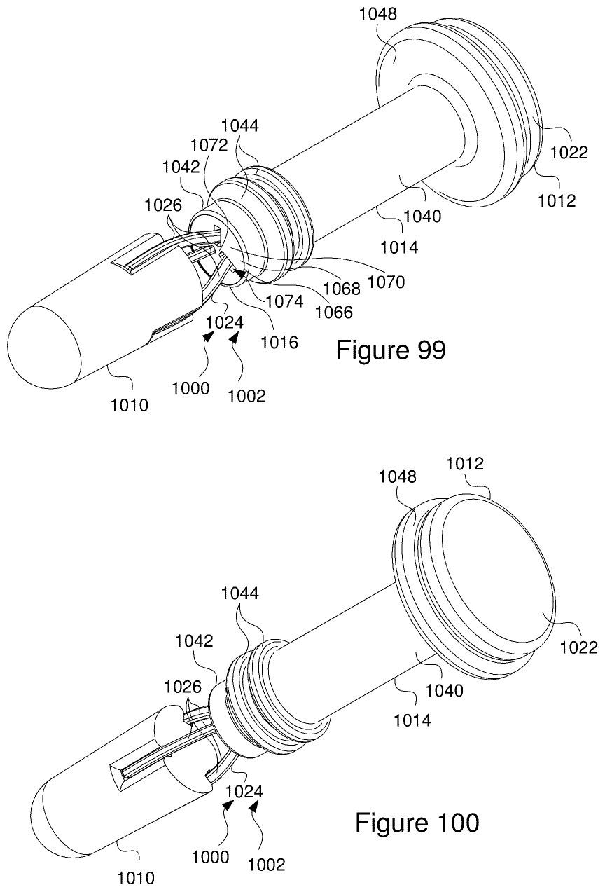

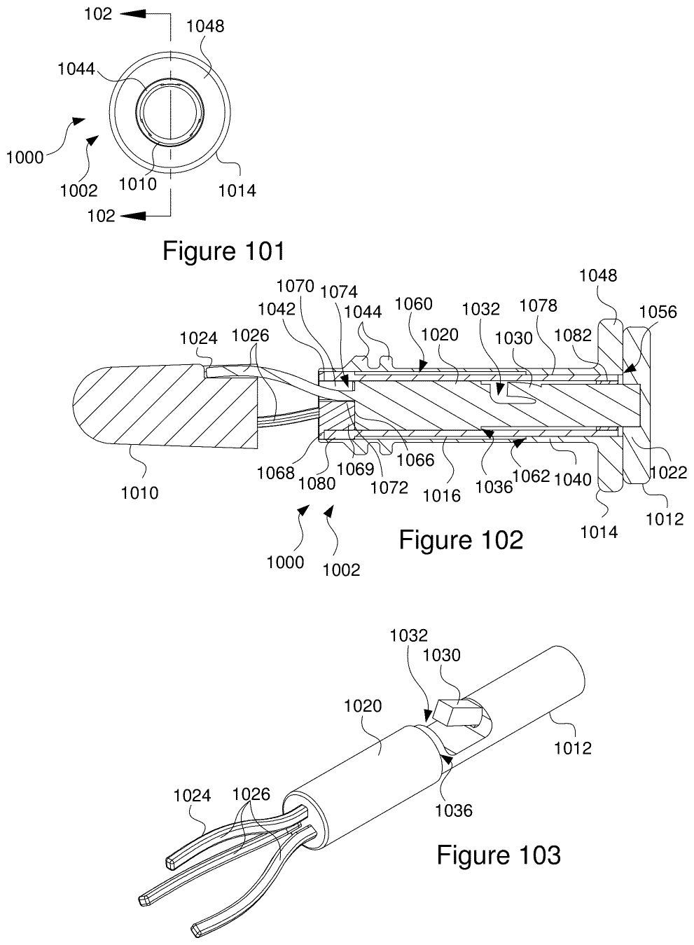

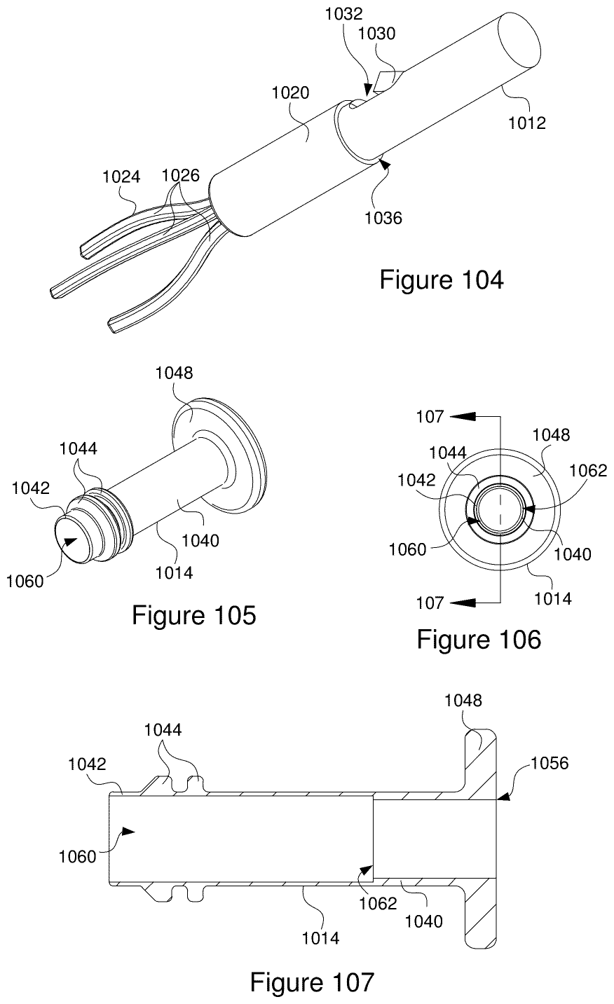

Referring to FIGS. 99-115, another earplug loading apparatus 1000 will be discussed. The earplug loading apparatus 1000 can have a stop with a sleeve as in the loading apparatus 900 discussed above, and the earplug loading apparatus 1000 may operate similarly to the earplug loading apparatus 900, but it may be structured differently, such as to include more jaws and to include other features instead of guides and slots of the loading apparatus 900. Specifically, the earplug loading apparatus 1000, which can include an earplug insertion device 1002, will be discussed. The earplug loading apparatus 1000 can include an earplug 1010, a loader 1012, a chamber 1014, and a stop 1016.

The loader 1012 can be structured to load the earplug 1010 (and/or some other earplug) into the chamber 1014 (i.e., at least partially in the chamber 1014, and possibly fully in the chamber 1014). The loader 1012 can also be structured to push the earplug 1010 out of the chamber 1014 and into the ear canal of the user (like the ear canal 106 of the user 102 discussed above) via the stop 1016.

As illustrated in FIGS. 102-104, the loader 1012 can include a loader body 1020 that can be sized to fit into the stop 1016, which can be sized to fit into the chamber 1014. For example, the loader body 1020 can be a generally cylindrical body. The loader body 1020 can extend from a loader base 1022 to a grip 1024, with a grip direction (which is also a distal direction in this example because the loader 1012 can be oriented the same way relative to the chamber 1014 for loading and inserting operations) being axially toward a terminus of the grip 1024 and a base direction (which is also a proximal direction in this example) being axially toward the loader base 1022. The loader base 1022 can be a separate part that is secured to a proximal end of the loader body 1020, such as with an adhesive or with one or more mechanical fasteners. The loader base 1022 can protrude radially out from the loader body 1020 to form a flange at the proximal end of the loader 1012.

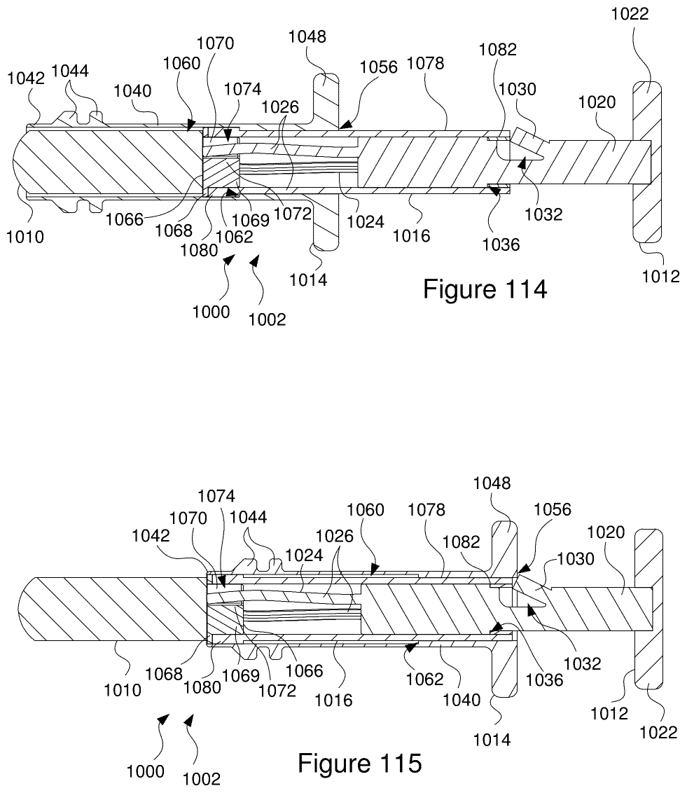

As an example, the grip 1024 can include three opposing jaws 1026 that extend from the loader body 1020 in a curved manner, like the jaws of the loader 512 discussed above, but with more jaws, and the jaws may be shaped somewhat differently. Alternatively, embodiments may include more than three jaws. As with the other loader bodies discussed herein, the loader body 1020 may be some other shape, such as where at least a portion of the loader body has a generally plus (+) shaped cross section or some other non-circular cross section instead of the circular cross section of a cylinder. The loader 1012 can also have a catch 1030, which can be a cantilever beam that extends from a wall of an aperture such as a cutout 1032 defined in the loader body 1020. The catch 1030 and the cutout 1032 can be like the catch and cutout of the loader 112 from the earplug loading apparatus 100 discussed above, but the catch 1030 can be structured to contact a proximal end of the stop 1016 as well as a proximal end of the chamber 1014 when the catch 1030 is extended (see FIGS. 114-115) and to fit within the stop 1016 and the chamber 1014 when the catch 1030 is depressed (see FIG. 102).

The loader 1012 can also include a protruding proximally facing shoulder 1036 that protrudes radially out from a smaller diameter proximal portion of the loader body 1020, with a larger diameter distal portion of the loader body 1020 extending distally from the proximally facing shoulder 1036. For example, the proximally facing shoulder 1036 may be located axially between the grip 1024 and the catch 1030.

Referring to FIGS. 105-107, the chamber 1014 can be like the chamber 714 illustrated in FIGS. 56-60 and described above. Like the chamber 714, the chamber 1014 can include a chamber body 1040, a distal tip 1042, distal tip flanges 1044, a base flange 1048, a catch area 1056 (see FIG. 107), and an axially extending larger diameter inner bore 1060 along its inner surface that can end in a distally facing shoulder 1062.

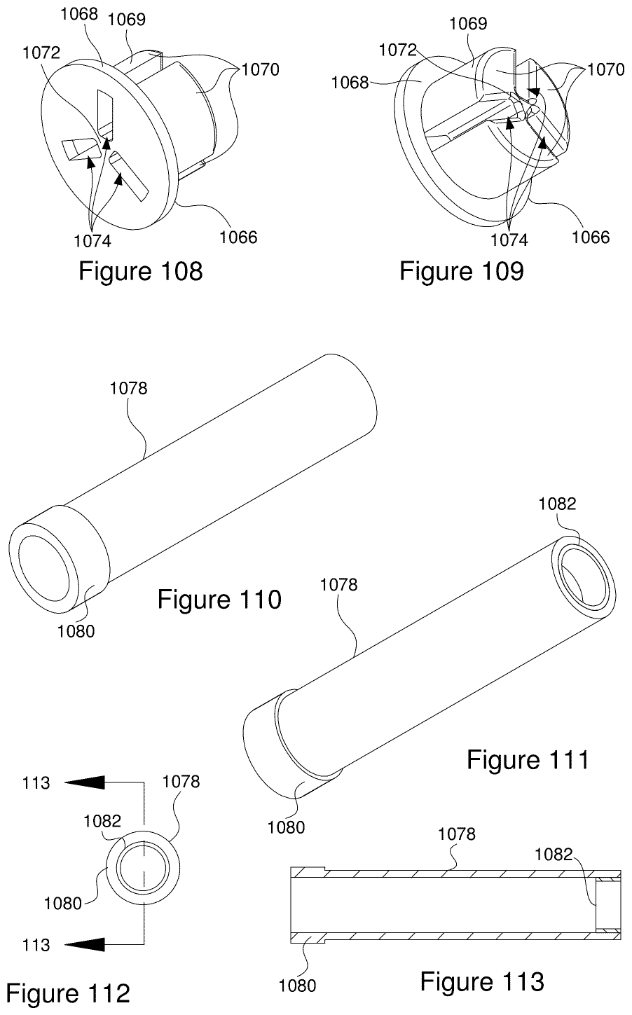

Referring to FIGS. 108-109, the stop 1016 can include a stop cap 1066, which can include a distal flange 1068 extending radially out in a ring from a distal end of a cap body 1069. The cap body 1069 can extend in the proximal direction from the distal flange 1068. The cap body 1069 can have a cross section that is generally in the shape of three truncated circle sectors that are joined together by a joining area at their inner portions. Also, generally rectangular gaps can be defined between the sectors. Thus, the cap body 1069 of the stop 1016 can include three circumferentially spaced sector areas 1070 formed by the general sector shapes of the cross section. The cap body 1069 can also include a joining wall 1072 joining the sector areas 1070. Thus, the cap body 1069 can define three access gaps extending 1074 between the sector areas 1070 radially out from the joining wall 1072, and extending radially in from the distal flange 1068 (the portions of the distal flange that extend around the access gaps 1074 between the sector areas 1070) to the joining wall 1072. The outer diameter of the sector areas 1070 can be sized relative to an inner diameter of a stop sleeve 1078 of the stop 1016 so that the sector areas 1070 can slide inside the stop sleeve 1078. Also, the access gaps 1074 can be sized and positioned to receive the jaws 1026 of the grip 1024 of the loader 1012, so that the jaws 1026 can slide through the access gaps 1074.

Referring to FIGS. 110-113, the stop sleeve 1078 can include a cylindrical tube body. The stop sleeve 1078 can also include a flange 1080 extending out from the stop sleeve 1078 at or near a distal end of the stop sleeve 1078.

The flange 1080 can have an outer diameter that is sized to allow the flange 1080 to slide along the inner bore 1060 of the chamber 1014, but to engage the distally facing shoulder 1062 of the chamber 1014 at the proximal end of the inner bore 1060 to inhibit further proximal direction movement of the stop sleeve 1078 inside the chamber 1014.

The distally facing shoulder 1062 of the chamber 1014 and a proximally facing end of the flange 1080 of the stop 1016 can be located at axial positions to allow the stop 1016 to be slid proximally far enough along the chamber 1014 to allow the earplug 1010 to be seated in the chamber 1014 between the stop 1016 and the distal tip 1042 of the chamber 1014, although some portion of the earplug 1010 may protrude out of the distal tip 1042 when the earplug 1010 is loaded in the chamber and seated against the stop 1016.

Thus, in use, the stop 1016 can slide within the chamber 1014 to a proximal position wherein the proximal end of the flange 1080 of the stop sleeve 1078 can contact the distally facing shoulder 1062 of the chamber 1014 (see FIG. 114) to inhibit further proximal direction movement of the stop 1016 relative to the chamber 1014.

The stop sleeve 1078 can also include an inner ring 1082, which can be secured to the body of the stop sleeve 1078 and can extend radially in from the body of the stop sleeve 1078 at a proximal end of the stop sleeve 1078. The stop cap 1066 can be joined with the stop sleeve 1078 to form the stop 1016, with the distal flange 1068 of the stop cap 1066 abutting a distal end of the stop sleeve 1078 and with the cap body 1069 of the stop cap 1066 extending in the proximal direction into the stop sleeve 1078. As an example, the stop cap 1066 can be joined to the cap body 1069 with an adhesive.

The loader 1012 can be positioned at least partially in the stop 1016, with the loader body 1020 able to slide axially relative to the stop 1016 between a proximal position in which the proximally facing shoulder 1036 of the loader 1012 can engage the inner ring 1082 of the stop 1016 (see FIGS. 114 and 115) and a distal position in which a distal end of the loader body 1020 abuts a proximal end of the stop cap 1066 (see FIG. 102). Thus, the engagements between the stop 1016 and the loader 1012 can inhibit relative axial movement between the stop 1016 and the loader 1012 beyond the proximal and distal positions, but allow relative axial movement between those positions. Also, when the loader 1012 is moved axially from the distal position to the proximal position, the catch 1030 can spring out to engage the proximal end of the stop 1016. Accordingly, in the proximal position of the loader 1012 relative to the stop 1016, the engagement of the catch 1030 with the stop 1016 can inhibit movement of the loader 1012 in the distal direction relative to the stop 1016 and the engagement of the proximally facing shoulder 1036 of the loader 1012 and the inner ring 1082 of the stop 1016 can inhibit movement of the loader 1012 in the proximal direction relative to the stop 1016. However, the catch 1030 can be depressed radially in by pressing on the catch 1030 to allow the catch 1030 to slide into the inner ring 1082 and further into the stop 1016, allowing the loader 1012 to move toward its distal position relative to the stop 1016.

Also, as noted above, the stop 1016 can slide within the chamber 1014 to a proximal position wherein the proximal end of the flange 1080 of the stop sleeve 1078 can contact the distally facing shoulder 1062 of the chamber 1014 (see FIG. 114). Moreover, the stop 1016 can slide within the chamber 1014 to a distal position wherein the catch 1030 of the loader 1012 can engage the catch area 1056 of the chamber 1014 to inhibit further axial movement of the loader 1012 or the stop 1016 in the distal direction relative to the chamber 1014. Depressing the catch 1030 from this position illustrated in FIG. 115 can allow the stop 1016 and the loader 1012 to move further axially in the distal direction. However, a user can push the stop 1016 in the proximal direction relative to the chamber 1014 to configure the earplug loading apparatus 1000 in the configuration illustrated in FIG. 102, where the jaws 1026 can extend out of the distal end of the stop 1016 and out of the distal end of the chamber 1014 to grasp the earplug 1010. Also, in an alternative embodiment, the inner ring 1082 may extend farther in the proximal direction and a proximal end of the inner ring 1082 of the stop 1016 can extend radially out to engage the catch area 1056 of the chamber 1014 to inhibit further movement of the stop 1016 in the distal direction relative to the chamber 1014 beyond the position illustrated in FIG. 102. For such an embodiment, when assembling the apparatus, the inner ring could be secured to the stop sleeve after the stop sleeve is inserted into the chamber.

In the proximal position of the loader 1012 relative to the stop 1016 illustrated in FIGS. 114-115, the jaws 1026 of the loader 1012 can be retracted into the stop 1016. When the loader 1012 is pulled in the proximal direction to the proximal position relative to the stop 1016, the catch 1030 can spring back out to its extended position to again inhibit distal direction movement of the loader 1012 relative to the stop 1016. Additionally, distal direction movement of the loader 1012 relative to the stop 1016 and the chamber 1014 can be stopped by engagement between a distal end of the loader body 1020 and a proximal end of the cap body 1069 of the stop cap 1066, as well as engagement between the loader base 1022 and the proximal ends of the stop 1016 and the chamber 1014 (see, e.g., FIG. 102).

Referring now to FIGS. 99-102 and FIGS. 114-115, use of the earplug loading apparatus 1000 to load the earplug 1010 into the chamber 1014 will be discussed. For loading, the loader 1012 and the chamber 1014 can be configured in the loading configuration of FIGS. 99-102, with the loader 1012 and the stop 1016 both in their distal positions relative to the chamber 1014. If the loader 1012 and the stop 1016 had been in their proximal positions as illustrated in FIG. 114, then to reach the distal positions, the loader 1012 and the stop 1016 could be pushed in the distal direction relative to the chamber 1014 to push the stop 1016 in the distal direction relative to the chamber 1014. The stop 1016 can stop in the distal position illustrated in FIG. 115. The catch 1030 could be depressed radially into the retracted position illustrated in FIG. 102, and the loader 1012 could be pushed in the distal direction relative to the chamber 1014 and the stop 1016. The loader 1012 could continue to be inserted in the distal direction relative to the chamber 1014 until the jaws 1026 of the grip 1024 of the loader 1012 slid through the access gaps 1074 of the stop 1016, with the distal portions of the jaws 1026 protruding out and beyond the distal tip 1042 of the chamber 1014, as illustrated in FIGS. 99-102. Such movement may include applying force in the proximal direction to the stop 1016 relative to the chamber 1014 to keep or move the stop 1016 in or to the position illustrated in FIGS. 99-102 relative to the chamber 1014. In this position, the sector areas 1070 of the stop 1016 can be positioned on opposite sides of each jaw 1026, and the wall 1072 joining the sector areas 1070 can be positioned between the jaws 1026. With the loader 1012 and the chamber 1014 in the position illustrated in FIGS. 99-102, the earplug 1010 can be inserted between the jaws 1026 of the loader 1012, as is illustrated in FIGS. 99-102.

With the jaws 1026 of the loader 1012 gripping the earplug 1010 as illustrated in FIGS. 99-102, the loader 1012 can be pulled in the proximal direction relative to the chamber 1014. As this happens, the chamber 1014 can squeeze the jaws 1026 inwardly toward each other as the jaws are pulled into the chamber 1014, tightening the grip of the jaws 1026 on the earplug 1010. Additionally, the jaws 1026 can pull the earplug 1010 into the chamber 1014. This proximal direction movement can also slide the stop 1016 in the proximal direction in the chamber 1014 until movement of the stop 1016 is stopped at the proximal position of the stop 1016 relative to the chamber 1014. The stop 1016 can in turn inhibit further proximal movement of the earplug 1010 along the chamber 1014. However, the loader 1012, including the jaws 1026, can continue to slide proximally relative to the chamber 1014 until the loader 1012 reaches its proximal position relative to the stop 1016 as illustrated in FIG. 114, with the earplug 1010 still loaded in the chamber 1014 in a position wherein a proximal end of the earplug 1010 can be seated against a distal end of the stop 1016.

Referring now to FIGS. 114-115, use of the earplug loading apparatus 1000 to push or plunge the earplug 1010 from the chamber 1014 and insert the earplug 1010 into an ear canal will be discussed. As illustrated in FIG. 114, the loader 1012 and the stop 1016 can each be in their proximal positions (with the stop 1016 in its proximal position relative to the chamber 1014 and the loader 1012 in its proximal position relative to the stop 1016). From there, with the distal tip 1042 of the chamber 1014 positioned in an opening of an ear canal, the loader 1012 and/or the stop 1016 can be pushed in the distal direction. If the loader 1012 is pushed, the catch 1030 of the loader 1012 can push the proximal end of the stop 1016 in the distal direction along with the loader 1012. As this happens, the stop 1016 and the loader 1012 can push the earplug 1010 in the distal direction relative to the chamber 1014. Thus, the loader 1012 can be used to push the earplug in the distal direction relative to the chamber 1014 via the stop 1016. This motion can continue until the stop 1016 reaches its distal position relative to the chamber 114 illustrated in FIG. 115 as it pushes the earplug 1010 out of the chamber 1014 and inserts the earplug 1010 into an ear canal of a user (see FIG. 115, with the ear canal not being illustrated). Thus, the loader 1012 can engage the earplug 1010 via the stop 1016 to push and eject the earplug 1010 from the chamber 1014 via the stop 1016. The engagement between the loader 1012 and the stop 1016 and the chamber 1014 can inhibit further movement of the stop 1016 beyond the distal position of the stop 1016 illustrated in FIG. 115 due to the catch 1030 of the loader 1012 engaging the chamber 1014 and the proximally facing shoulder 1036 of the loader 1012 engaging the stop 1016.

The loader 1012 can be used to load additional earplugs into the chamber 1014 in the same manner described above for the earplug 1010, and such earplugs can be plunged from the chamber and inserted into an ear canal in the same manner as described above for the earplug 1010.

The components of the earplug loading apparatus 1000 can be made of materials that are appropriate for the functions and structures described herein. For example, the loader 1012, the stop 1016, and the chamber 1014 may be made of polymer materials, composite materials, and/or metals. For example, the materials may be appropriately strong, stiff/flexible, and durable. The loader 1012, components of the stop 1016, and/or the chamber 1014 can be formed with manufacturing techniques such as molding, milling, lathing, and/or additive manufacturing such as 3D printing. As with other earplugs discussed herein, the earplug 1010 can be made of a compliant material such as foam, and such earplugs can be formed by manufacturing processes such as molding.

To assemble the earplug loading apparatus 1000, the loader 1012 without its loader base 1022 can be inserted into the stop sleeve 1078 (with the stop sleeve's inner ring 1082 secured in place). Specifically, the loader 1012 and the stop sleeve 1078 can be oriented with their distal and proximal ends oriented as illustrated in the figures and discussed above, and the proximal end of the loader body 1020 can be inserted into the distal end of the stop sleeve 1078. With the loader 1012 positioned in the stop, the stop cap 1066 can be inserted into the distal end of the stop sleeve 1078 with the access gaps 1074 of the stop cap 1066 aligned with the jaws 1026 of the loader 1012. The stop cap 1066 can be secured in place in the stop sleeve 1078, such as with an adhesive, to form the stop 1016. The resulting stop-loader subassembly can be inserted into the distal end of the chamber 1014 until the proximal end of the loader body 1020 extends out of the proximal end of the chamber 1014. The loader base 1022 can then be secured on the proximal end of the loader body 1020, such as with an adhesive, to form the loader 1012, and any adhesives that are used for assembly can be allowed to cure.

Alternatively, some or all of the components of the earplug loading apparatus 1000 can be formed already assembled using additive manufacturing techniques such as 3D printing.

### VIII. Example Different Earplug Orientation

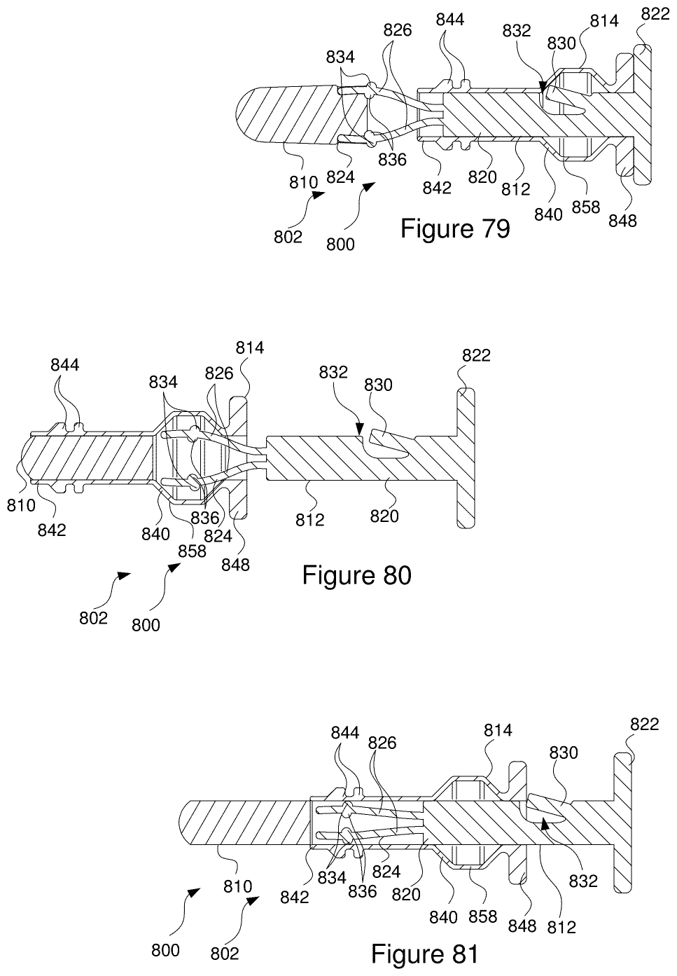

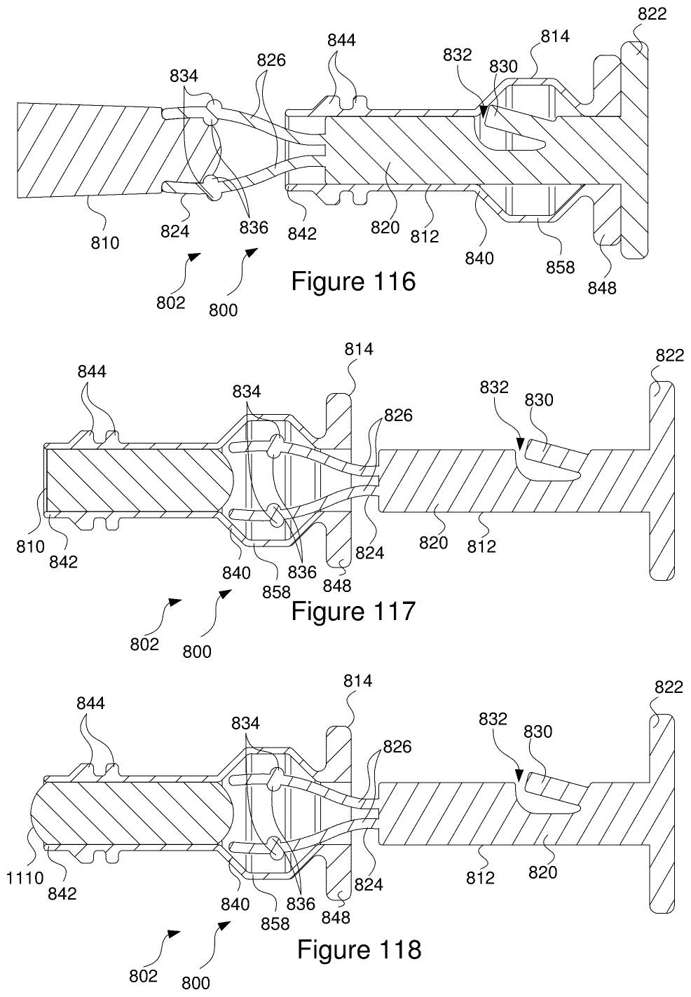

In the embodiments illustrated in the figures discussed above, the earplug includes a flat end and a rounded end, and the earplug is oriented with the rounded end facing in the distal direction, so that the rounded end would be inserted first into the ear canal. However, the earplug can be oriented the opposite way with the rounded end of the earplug facing in the proximal direction so that the flat end of the earplug would proceed before the rounded end when the earplug is pushed from the chamber and into the ear canal. Such an orientation is illustrated in FIGS. 116-117, which illustrate the earplug loading apparatus 800 as in FIGS. 79-80 above. The earplug loading apparatus 800 could be structured and could operate in the same manner with the earplug in the orientation of FIGS. 116-117 or the orientation of FIGS. 79-80. This earplug orientation of FIGS. 116-117 could also be used with other earplug loading apparatuses discussed above. Also, with any of the earplug loading apparatuses, they may be used with earplugs having different shapes, such as generally cylindrical earplugs with both ends being rounded as illustrated in FIG. 118 with an earplug 1110, which may be a foam earplug as with other examples of earplugs discussed above.

### IX. Aspects of the Disclosure

Features discussed in each of the aspects below may be utilized by themselves and/or combined with each other in any workable combination not precluded by the discussion herein, including combining features from a technique discussed with reference to one aspect with features discussed with reference to a different aspect. Also, structural features such as apparatuses, devices, and/or components may include means for performing each of the acts discussed in the context of techniques, by themselves and/or in different combinations.

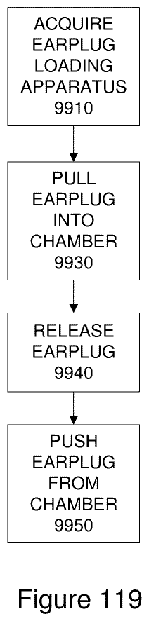

Referring to FIG. 119, according to one aspect, a technique can include acquiring 9910 an earplug loading apparatus. The earplug loading apparatus can include a chamber of an earplug insertion device and a loader. The technique can further include pulling 9930 the earplug at least partially into the chamber. The pulling 9930 can include using the loader to pull the earplug relative to the chamber.

The pulling 9930 of the earplug can include holding the earplug using the loader and moving the loader relative to the chamber while the loader holds the earplug. The holding of the earplug can include holding the earplug with jaws of the loader. The jaws of the loader may squeeze together to hold the earplug more firmly as the jaws of the loader enter the chamber.

The technique can further include releasing 9940 a hold of the loader on the earplug while the earplug is positioned at least partially in the chamber. The releasing 9940 can include jaws of the loader springing outward while the jaws exit the chamber. The releasing 9940 can include using a stop positioned at least partially inside the chamber to inhibit movement of the earplug while a grip of the loader is moved away from the earplug. The stop can include a sleeve, and at least a portion of the loader can slide within the sleeve when releasing a hold of the loader on the earplug.

The technique can further include pushing 9950 the earplug from the chamber. For example, the pushing of the earplug from the chamber can include using the loader as a plunger to push the earplug from the chamber. Alternatively, the pushing 9950 may use a separate plunger that is not part of the loader.

At least a portion of the loader can pass at least partially through the chamber ahead of the earplug as the earplug is being pulled at least partially into the chamber.

The chamber can include an expanded section, and the technique can further include expanding a grip of the loader in the expanded section to loosen a grasp of the grip on the earplug and release 9940 the earplug from the grip.

According to another aspect, an earplug loading apparatus can include a loader including a grip that is structured to pull an earplug at least partially into a chamber of an earplug insertion device.

The grip can be structured to hold the earplug while the grip pulls the earplug at least partially into the chamber. The grip may be at least partially retractable into a body of the loader.

The earplug loading apparatus may further include the earplug insertion device, and the earplug insertion device may be configured to hold the earplug in position in the chamber and to push the earplug from the chamber into an ear canal. The earplug loading apparatus may also include the earplug, and possibly additional earplugs that can be used with the earplug loading apparatus.

The grip of the loader can include jaws extending from a body of the loader For example, the jaws may include two jaws, and the jaws may include three or more jaws.

The loader can be structured to pull the earplug at least partially into the chamber while the grip is holding the earplug. The loader can be further structured to push the earplug from the chamber and into an ear canal.

The loader can include a stop that is structured to inhibit movement of the loader past a predetermined position when pushing the earplug from the chamber into an ear canal. The stop can include a sleeve that is structured so that at least a portion of the loader is slidable within the sleeve when releasing a hold of the loader on the earplug. At least one of the stop and the loader can be moveable between a stopped position of the loader relative to the stop where the stop inhibits movement of the loader past a predetermined position relative to the chamber when pushing the earplug from the chamber into an ear canal and a non-stopped position wherein the stop does not inhibit movement of the loader past the predetermined position relative to the chamber when pushing the earplug from the chamber into an ear canal.

The chamber can include an expanded section, and the apparatus can be structured to expand a grip of the loader in the expanded section to loosen a grasp of the grip on the earplug and release the earplug from the grip.

The loader can include jaws that are structured to grasp the earplug when pulling the earplug into the chamber. One or more of the jaws can include a protrusion that protrudes radially outward from the jaw in a direction away from the earplug when the earplug is grasped by the one or more jaws (such as the outwardly protruding nubs discussed above). Also, one or more of the jaws can include a protrusion that protrudes radially inward from the jaw in a direction toward the earplug when the earplug is grasped by the one or more jaws, and the protrusion may aid in positioning the earplug in a predetermined axial position with at least part of the earplug being between the jaws (such as with the inwardly protruding nubs discussed above).

The earplug can include two opposite ends that are both rounded. Or the earplug can have one rounded end and one flat end opposite the rounded end, and the loading of the earplug can include loading the earplug so that the earplug is oriented to be pushed out of the chamber with the flat end proceeding before the rounded end.

According to another aspect, an earplug loading apparatus can include an earplug insertion device that includes a chamber. The earplug loading apparatus can further include a loader that is structured to load an earplug at least partially into the chamber of the earplug insertion device. The loader can be structured to be positioned through the chamber of the earplug insertion device with at least a portion of the loader extending from opposite ends of the chamber at the same time.

The loader may include a grip that can be structured to hold the earplug and pull the earplug at least partially into the chamber of the earplug insertion device.

The earplug loading apparatus can include the chamber and a stop in the chamber (i.e., at least partially in the chamber). The stop can be structured to inhibit movement of the earplug beyond a predetermined position in the chamber. The stop can be slidable within the chamber, and the stop can be structured to push an earplug from the chamber in an earplug insertion operation of the earplug insertion device.

The subject matter defined in the appended claims is not necessarily limited to the benefits described herein. A particular implementation of the invention may provide all, some, or none of the benefits described herein. Although operations for the various techniques are described herein in a particular, sequential order for the sake of presentation, it should be understood that this manner of description encompasses rearrangements in the order of operations, unless a particular ordering is required. For example, operations described sequentially may in some cases be rearranged or performed concurrently. Techniques described herein with reference to flowcharts may be used with one or more of the systems described herein and/or with one or more other systems. Moreover, for the sake of simplicity, flowcharts may not show the various ways in which particular techniques can be used in conjunction with other techniques.

While particular embodiments are discussed above, it will be understood by those skilled in the art that various changes in form and details may be made therein without departing from the spirit and scope of the invention as defined by the claims.

I/We claim:

1. A method comprising:
acquiring an earplug loading apparatus comprising a chamber of an earplug insertion device and a loader; and
pulling an earplug at least partially into the chamber of the earplug insertion device, the pulling of the earplug comprising using the loader to pull the earplug relative to the chamber.

2. The method of claim 1, wherein the pulling of the earplug comprises holding the earplug using the loader and moving the loader relative to the chamber while the loader holds the earplug.

3. The method of claim 2, wherein holding the earplug comprises holding the earplug with jaws of the loader.

4. The method of claim 3, wherein the jaws of the loader squeeze together to hold the earplug more firmly as the jaws of the loader enter the chamber.

5. The method of claim 2, further comprising releasing a hold of the loader on the earplug while the earplug is positioned at least partially in the chamber.

6. The method of claim 5, wherein the releasing of the hold of the loader on the earplug comprises jaws of the loader springing outward while the jaws exit the chamber.

7. The method of claim 5, wherein the releasing of the hold of the loader on the earplug comprises using a stop positioned at least partially inside the chamber to inhibit movement of the earplug while a grip of the loader is moved away from the earplug.

8. The method of claim 7, wherein the stop comprises a sleeve and wherein at least a portion of the loader slides within the sleeve when releasing a hold of the loader on the earplug.

9. The method of claim 1, further comprising pushing the earplug from the chamber.

10. The method of claim 9, wherein the pushing of the earplug from the chamber comprises using the loader as a plunger to push the earplug from the chamber.

11. The method of claim 1, wherein at least a portion of the loader passes at least partially through the chamber ahead of the earplug as the earplug is being pulled at least partially into the chamber.

12. The method of claim 1, wherein the chamber comprises an expanded section, and wherein the method further comprises expanding a grip of the loader in the expanded section to loosen a grasp of the grip on the earplug and release the earplug from the grip.

13. The method of claim 12, wherein the loader comprises jaws that are structured to grasp the earplug when pulling the earplug into the chamber, and wherein one or more of the jaws comprises a protrusion that protrudes radially outward from the jaw in a direction away from the earplug when the earplug is grasped by the one or more of the jaws.

14. The method of claim 12, wherein the loader comprises jaws that are structured to grasp the earplug when pulling the earplug into the chamber, and wherein one or more of the jaws comprises a protrusion that protrudes radially inward from the jaw in a direction toward the earplug when the earplug is grasped by the one or more of the jaws, and wherein the protrusion aids in positioning the earplug in a predetermined axial position with at least part of the earplug being between the jaws.

15. The method of claim 1, wherein the earplug includes two opposite ends that are both rounded.

16. The method of claim 1, wherein the earplug has one rounded end and one flat end opposite the rounded end, and wherein the loading of the earplug comprises loading the earplug so that the earplug is oriented to be pushed out of the chamber with the flat end proceeding before the rounded end.

17. An earplug loading apparatus comprising:
a loader comprising a grip that is structured to pull an earplug at least partially into a chamber of an earplug insertion device.

18. The earplug loading apparatus of claim 17, wherein the grip is structured to hold the earplug while the grip pulls the earplug at least partially into the chamber.

19. The earplug loading apparatus of claim 17, wherein the grip is at least partially retractable into a body of the loader.

20. The earplug loading apparatus of claim 17, further comprising the earplug insertion device, the earplug insertion device being configured to hold the earplug in position in the chamber and to push the earplug from the chamber into an ear canal.

21. The earplug loading apparatus of claim 20, further comprising the earplug.

22. The earplug loading apparatus of claim 17, wherein the grip comprises jaws extending from a body of the loader.

23. The earplug loading apparatus of claim 22, wherein the jaws comprise three or more jaws.

24. The earplug loading apparatus of claim 17, wherein loader is structured to pull the earplug at least partially into the chamber while the grip is holding the earplug.

25. The earplug loading apparatus of claim 17, wherein the loader is further structured to push the earplug from the chamber into an ear canal.

26. The earplug loading apparatus of claim 17, wherein the loader comprises a stop that is structured to inhibit movement of the loader past a predetermined position when pushing the earplug from the chamber into an ear canal.

27. The earplug loading apparatus of claim 26, wherein the stop comprises a sleeve that is structured so that at least a portion of the loader is slidable within the sleeve when releasing a hold of the loader on the earplug.

28. The earplug loading apparatus of claim 26, wherein at least one of the stop and the loader are moveable between a stopped position of the loader relative to the stop where the stop inhibits movement of the loader past a predetermined position relative to the chamber when pushing the earplug from the chamber into an ear canal and a non-stopped position wherein the stop does not inhibit movement of the loader past the predetermined position relative to the chamber when pushing the earplug from the chamber into an ear canal.

29. The earplug loading apparatus of claim 17, wherein the chamber comprises an expanded section, and wherein the earplug loading apparatus is structured to expand a grip of the loader in the expanded section to loosen a grasp of the grip on the earplug and release the earplug from the grip.

30. An earplug loading apparatus comprising:
a chamber of an earplug insertion device; and
a loader that is structured to load an earplug at least partially into the chamber of the earplug insertion device, the loader being structured to be positioned through the chamber of the earplug insertion device with at least a portion of the loader extending from opposite ends of the chamber at the same time.

31. The earplug loading apparatus of claim 30, wherein the loader comprises a grip that is structured to hold the earplug and pull the earplug at least partially into the chamber of the earplug insertion device.

32. The earplug loading apparatus of claim 30, wherein the earplug loading apparatus comprises the chamber and a stop in the chamber, the stop being structured to inhibit movement of the earplug beyond a predetermined position in the chamber.

33. The earplug loading apparatus of claim 32, wherein the stop is slidable within the chamber and the stop is structured to push an earplug out of the chamber in an earplug insertion operation of the earplug insertion device.
